(19) United States
(12) Patent Application Publication
(10) Pub. No.: US 2021/0225518 A1
(43) Pub. Date: Jul. 22, 2021

(54) TEXT-BASED ANALYSIS TO COMPUTE LINGUISTIC MEASURES IN-SITU TO AUTOMATICALLY PREDICT PRESENCE OF A COGNITIVE DISORDER BASED ON AN ADAPTIVE DATA MODEL

(71) Applicant: CueBack Technology, Inc., Palo Alto, CA (US)

(72) Inventors: Arnold Papp, Palo Alto, CA (US); Daniel Papp, Palo Alto, CA (US); Michael Papp, Palo Alto, CA (US)

(73) Assignee: CueBack Technology, Inc., Palo Alto, CA (US)

(21) Appl. No.: 17/164,568

(22) Filed: Feb. 1, 2021

Related U.S. Application Data
(63) Continuation-in-part of application No. 15/961,432, filed on Apr. 24, 2018.
(60) Provisional application No. 62/490,401, filed on Apr. 26, 2017.

(51) Int. Cl. G16H 50/30; A61B 5/00; G06F 40/10; G16H 50/20; G16H 20/70

(52) U.S. Cl. CPC G16H 50/30; A61B 5/4088; A61B 5/7275; A61B 5/7475; G16H 20/70; A61B 5/7246; A61B 5/7264; G06F 40/10; G16H 50/20; A61B 5/4842

(57) ABSTRACT

Various embodiments relate generally to data science, data analysis, and computer software and systems to apply psychological science and principles to provide an interface, as one of a number of sources of text, for facilitating memory recall, and, more specifically, to a computing and data storage platform that facilitates recall of one or more memories collaboratively and performs in-situ text-based analysis automatically to calculate one or more linguistic measures to predict a cognitive disorder or deficiency. In some examples, a method may include receiving data representing text, classifying words based on a syntactic function to analyze the text, identifying portions of sentences and identifying a source of the text. In some cases, the method includes executing instructions to characterize text and portions of sentences, the characterized data being indicative of a cognitive state or a deficiency thereof.

### CROSS-REFERENCE TO APPLICATIONS

This application is a continuation-in-part application of U.S. patent application Ser. No. 15/91,432 filed on Apr. 24, 2018 and titled “CUE DATA MODEL IMPLEMENTATION FOR ADAPTIVE PRESENTATION OF COLLABORATIVE RECOLLECTIONS OF MEMORIES,” which claims the benefit of U.S. Provisional Patent Application No. 62/490,401 filed on Apr. 26, 2017 and titled “ADAPTIVE PRESENTATION OF COLLABORATIVE RECOLLECTIONS OF MEMORIES,” all of which are herein incorporated by reference in its entirety for all purposes.

### FIELD

Various embodiments relate generally to data science, data analysis, and computer software and systems to apply psychological science and principles to provide an interface, as one of a number of sources of text, for facilitating memory recall, and, more specifically, to a computing and data storage platform that facilitates recall of one or more memories collaboratively and performs in-situ text-based analysis automatically to calculate one or more linguistic measures with which to predict a cognitive disorder or deficiency.

### BACKGROUND

Alzheimer's disease is a fatal neurodegenerative disease that currently affects about 5.8 million Americans and many others throughout the world. Characterized by a buildup of plaques and protein tangles in the brain, which causes neuron destruction, Alzheimer's disease primarily affects the memory, behavior, and other cognitive regions of the brain. Though brain degeneration caused by Alzheimer's disease is not reversible, early detection may prolong the life of those afflicted with Alzheimer's disease. In some cases, a life may be prolonged by as much as 20 years. Notably, according to some studies, approximately 5% of those persons diagnosed with Alzheimer's disease may be due to genetics. However, Alzheimer's disease in the other 95% may be caused by any number of known or unknown factors.

Advances in computing hardware and software have been applied to recent explorative studies to correlate language production (i.e., via text-based language) to cognitive disorders, such as Alzheimer's disease. Based on correlations between restricted samples of written language and a presence of a cognitive disorder, computer-based analysis of text-based language have been used to predict a likelihood that a patient may be afflicted with an onset of a cognitive disorder, such as the onset of Alzheimer's disease.

While traditional applications of computing systems to detect cognitive degenerative diseases are functional for some applications and/or patients, they are not well-suited to detect early onset of Alzheimer's disease or the like over a broader population sample. For example, conventional approaches to generating text samples with which to use in analysis are typically derived from manually-transcribed studies in a clinical environment, which usually skews data since elderly patients may be nervous and under an amount of stress to perform. Furthermore, some traditional approaches restrict the content of the samples of language production to a limited number of semantic categories (e.g., a limited number of words).

In some typical approaches, participants in cognitive studies are generally limited to interviews as a source of text (e.g., whether written or transcribed from oral collection), the interviews being a common source of language production samples. Further, conventional evaluations using a computing technologies are generally limited to language produced only be a participant when evaluating that participant's cognitive abilities.

Thus, what is needed is one or more solutions for facilitating detection, evaluation, and/or treatment of cognitive disorders, without the limitations of conventional techniques.

### BRIEF DESCRIPTION OF THE DRAWINGS

Various embodiments or examples (“examples”) of the invention are disclosed in the following detailed description and the accompanying drawings:

FIG. 1A is a diagram depicting an example of a collaborative recollection engine configured to analyze linguistic samples originating from one or more sources, according to some embodiments;

FIG. 1B is a diagram depicting an example of an application stack configured to implement applications to predict a cognitive state based on text-based analysis, according to some examples;

FIG. 2 is a diagram depicting an example of a linguistic engine configured to predict, detect, evaluate, and/or identify likelihood of a presence of a cognitive disorder, according to some examples;

FIGS. 3 and 4 are functional block diagrams depicting examples of the functionalities of a linguistic engine, according to at least one embodiment;

FIG. 5 is a diagram depicting a flow diagram as an example of analyzing linguistic samples to predict whether a cognitive deficiency may affect formation of a linguistic structure associated with language production, according to some embodiments;

FIG. 6 is an example of a user interface to receive one or more prompts to identify one or more user attributes that may be relevant to determine a cognitive state, according to some examples;

FIG. 7 is an example of a linguistic engine configured to analyze portions of language to determine user attributes relevant to ascertain a cognitive state, according to some examples;

FIG. 8 is an example of a user interface to analyze data entry of a social media page to identify attributes to evaluate cognitive abilities of a user, according to some examples;

FIG. 9 is a diagram depicting an example of a probabilistic prediction model to infer a cognitive state as a function of characterizing linguistic samples, according to some examples;

FIG. 10 is a diagram depicting an example of a computing platform configured to implement a linguistic engine to detect and manage instances of a challenged term, according to some examples; and

FIG. 11 illustrates examples of various computing platforms configured to provide various functionalities to components of a collaborative recollection engine and/or a linguistic engine, according to various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims, and numerous alternatives, modifications, and equivalents thereof. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

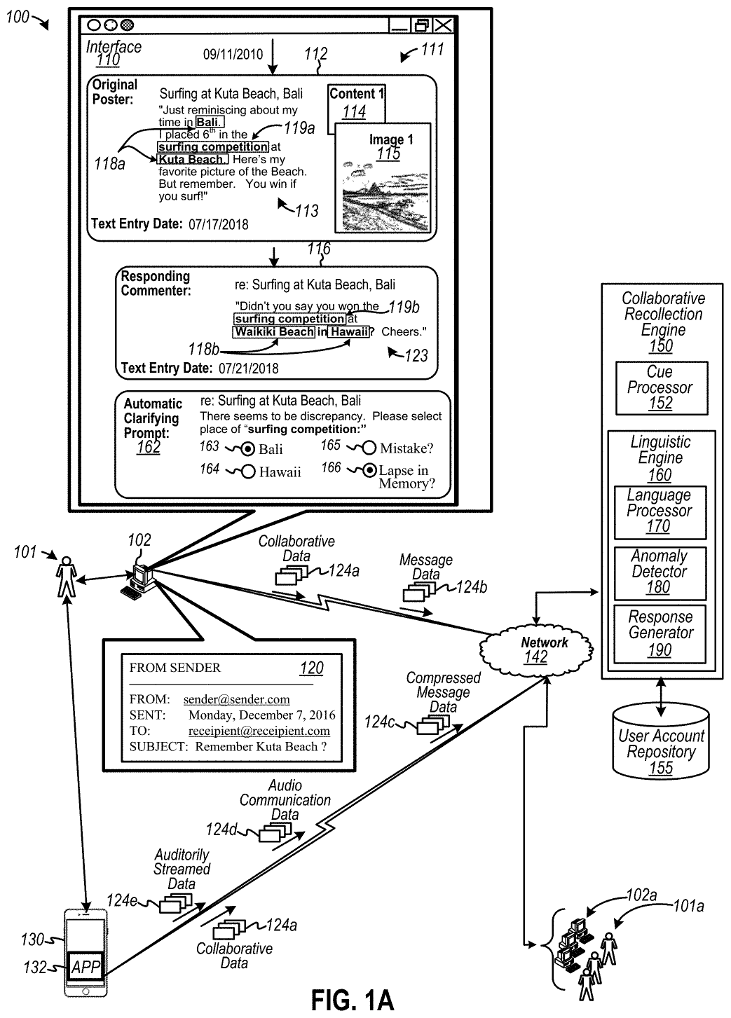

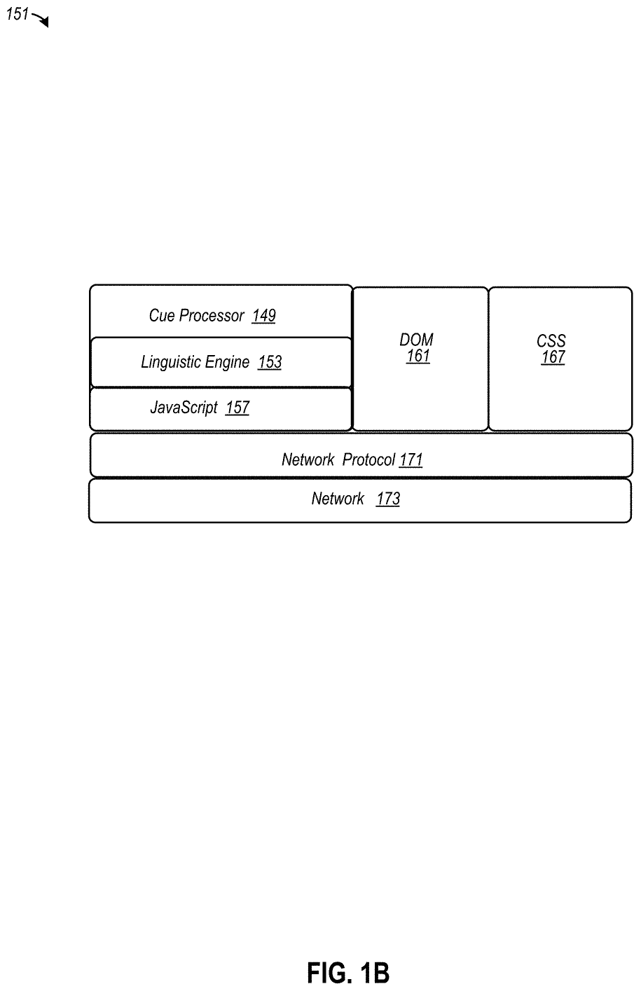

FIG. 1A is a diagram depicting an example of a collaborative recollection engine configured to analyze linguistic samples originating from one or more sources, according to some embodiments. Diagram 100 depicts an example of a collaborative recollection engine 150 configured to facilitate recollection of one or more memories of one or more users, and further configured to analyze data representing linguistic samples originating from one or more sources, including user interface 110 of computing device 102, to detect and evaluate presence of a cognitive disorder that may influence (e.g., detrimentally) formation of a text-based message, a verbal message, and the like. As shown, a user 101 may access a computing device 102 to interact via a network 142 with collaborative recollection engine 150. Diagram 100 also shows that collaborative recollection engine 150 may include a cue processor 152 and a linguistic engine 160, which, in turn, may include a language processor 170, and an anomaly detector 180, and a response generator 190, one or more of which may be implemented to cause generation of a collaborative recollection interface 110 at, for example, a computing device 102 or a mobile computing device 130 associated with a user 101.

Cue processor 152 may be configured to generate cues as “text”-based cues (e.g., news stories, archived emails or text messages, literary and written media, including books, etc.), “image”-based cues (e.g., photographs or other static imagery, animated imagery, video, etc.), “audio”-based cues (e.g., music, songs, sound effects, etc.), or any other type of medium in which stimuli may be presented to a user to evoke a memory or recollection. In other examples, cue processor 152 may also be configured to generate cues to clarify, for example, potential inconsistencies in linguistic samples over time (e.g., factual differences that may arise due to cognitive decline). In one example, a cue processor 152 may be configured to detect an inconsistency between recollections or text entries, such as describing different wedding anniversary dates or events at different points in time (e.g., which may be indicative of cognitive decline). In some cases, cue processor 152 may be configured to prompt user 101 to clarify accuracy of an anniversary date and may generate a prompt for presentation in interface 110 to request a response from user 101 indicative of whether the discrepancy was due to a simple mistake or a lapse in memory.

Cue processor 152 may be configured to determine and generate data representing one or more memory cues (or memory triggers) that may be configured to trigger recollection of a memory, regardless of whether the memory is recalled consciously, subconsciously, involuntarily, etc., or regardless of whether the memory is based on olfactory recollective memories (e.g., a scent of a spring morning after rainfall), visual recollective memories (e.g., a vivid recollection of a turquoise ocean near white sandy beaches of a Caribbean island), auditory recollective memories (e.g., a certain song or musical score), tactile or haptic recollective memories, etc. Types of memory cues may include data representing notable events, music, and popular cultural phenomenon, such as television (“TV”) shows and soundtracks, movies and corresponding soundtracks, commercials (e.g., TV, radio, etc.), sporting events, news events and stories, Internet-related events (e.g., memes), etc., or any other information likely to elicit or otherwise bolster a memory.

Cue processor 152 may be configured to generate cues independent of a user's actions (i.e., independent of user 101), and, as such, user 101 need not have to explicitly request for a cue to be created. For example, cue processor 152 may identify one or more attributes of user 101 (e.g., based on demographic data, including age, gender, etc., as well as associated family member identities and attributes, friend identities and attributes, as well as interests, such as sports, geographic locations to which user 101 has traveled, and many other attributes that may be used (e.g., as metadata) to identify cues for presentation to user 101 to elicit relevant memories to either recollection 112 or recollection 116, or both. Based on the one or more attributes of user 101, may be configured to generate cues as “text”-based cues (e.g., news stories, archived emails or text messages, literary and written media, including books, etc.), “image”-based cues (e.g., photographs or other static imagery, animated imagery, video, etc.), “audio”-based cues (e.g., music, songs, sound effects, etc.), or any other type of medium in which stimuli may be presented to a user to evoke a memory or recollection. In accordance with at least one implementation, cue processor 152 and/or other components of collaborative recollection engine 150 may be implemented in accordance with one or more examples described in U.S. patent application Ser. No. 15/961,432, which is incorporated herein by reference.

According to various examples, linguistic engine 160 may include software or hardware, or any combination of both, that may be configured to compute and detect anomalies in linguistic samples to predict a likelihood of cognitive deficiencies of one or more users 101 and 101a. In the example shown, language processor 170 may be configured to receive data representing language, such as data representing alpha-numeric text that constitutes a linguistic sample (e.g., text-based entry 112), and to further characterize one or more terms or words as well as one or more subsets of words constituting portions of sentences. Anomaly detector 180 may be configured to compute any number of linguistic measures or attributes based on a linguistic sample to determine whether one or more of the linguistic measures or attributes correlate to, for example, boundary or threshold values indicative of healthy users (e.g., persons with reduced or negligible cognitive deficiencies), or otherwise. Should a cognitive state be determined to be anomalous (e.g., non-healthy or non-normal), anomaly detector 180 may be further configured to predict identification of a type of cognitive disorder (e.g., Alzheimer's disease, Korsakoff syndrome, chronic traumatic encephalopathy (“CTE”), or any other disorder, according to some examples. Response generator 190 may generate an electronic message as a notification to medical personal, family members, or to computing system, whereby the electronic message may describe one or more cognitive states (e.g., one or more cognitive disorders).

According to various examples, collaborative recollection engine 150 may be configured to generate numerous amounts of sensory stimuli (e.g., visually, such as a website, auditorily, such as music, songs, spoken word, etc., olfactory-related stimuli, such as aromas, etc.) to facilitate a user's recollection of a memory, and to encourage user 101 to engage with, for example, interface 110 to write or otherwise provide a linguistic sample. Collaborative recollection engine 150 may be configured to implement functions configured to apply psychological techniques, which may be adapted in accordance with various functions and/or structures described herein, to evoke memory recall to assist in memory recollection, and to determine degeneration or deficiencies in memory over time (e.g., as a user ages). Various functions and structures described herein may be configured to assist memory access (e.g., episodic memory), whereby memories associated with encoded senses, such as sight, sound, smell, touch, taste, etc., may be readily accessible or relatable to a specific recollection of one or more instances of memories, or representations of a memory itself. An instance of memory, for example, may refer to recollection of a past experience associated with one of a number of encoded senses (e.g., a unit of memory). One or more instances of memory may be combined to constitute an episodic memory, such as a sequence of past experiences (e.g., a memory of performing an activity, such as snorkeling or surfing). While memories, such as episodic memories, may be consciously or explicitly recalled, some memories may be recalled subconsciously or responsively, for example, to one or more memory cues.

Accordingly, collaborative recollection engine 150 may be configured to generate stimuli that may supplement, refresh, and/or reconstitute one or more memories based on one or more memory cues or the like. Collaborative recollection engine 150 may be configured to facilitate memorialization or archival of a user's experiences (and usage of words and language to describe said experiences for linguistic analysis), with optional authorized access provided to other users to view, supplement, modify or otherwise enhance or even challenge a user's recollection of its experiences. As shown, collaborative recollection engine 150 may accept input via network 142 as collaborative data 124a from computing device 102 to form and relate data representing past experiences (and associated characteristics thereof) of user 101. Therefore, user 101 may be able to memorialize its memories as data configured for presentation or display in a text-based timeline 111 in interface 110. Diagram 100 depicts the stored and interrelated past experiences of user 101 being presented as timeline 111, which presents recollective memories of user 101. Timeline 111 is shown, in this example, to include a display of a recollection 112 describing the various facets of a past experience entitled “Surfing at Kuta Beach, Bali,” which is shown as a textual description 113 of a perspective and past experience of user 101. Recollection 112 may be supplemented by additional sources of stimuli provided by user 101 or any of users 101a, such as image 115 (e.g., digital photographs, or the like) and other content 114 (e.g., videos, sounds, music, etc.).

Therefore, collaborative recollection engine 150 may be configured to enrich recollection 112 collaboratively, so as to enable user 101 and users 101a to collaborate to bolster and fortify user's 101 recollection of its past experiences. According to some embodiments, user 101 and each of users 101a may be associated with user account identifiers that uniquely identify each of the users and may store data for facilitating one or more functionalities described or otherwise performed by collaborative recollection engine 150. As shown, user account repository 155 may store data representing user account information for each specific user 101 and users 101a or any other data including, but not limited to, data associated with linguistic samples and analyses of a user's cognitive state. As such, user 101 may identify user account identifiers (e.g., directly or indirectly) to grant access privileges to view one of recollections 112. Thus, some users 101a may not have access to recollections 112 to preserve privacy.

Note that in some cases, the term “collaborative” may include any data originating from one or more users 101 and 101a, as well as any related content (e.g., additional or modified text, images, audio, content, etc.), and the system (e.g., additional or modified text, images, audio, content, etc.) that may facilitate aggregation of encoded sensory stimuli (e.g., units of memory) to form data representing a recollection, which may form or be recollected as an episodic memory, autobiographical memory, etc. Also, “collaborative” may refer to data representing a resultant collaborative memory recollection based on conscious or unconscious recollections of constituent memories based on autobiographical memory of user 101, and also based on memory recollections of one or more other users 101a via one or more computing devices 102a. In some examples, collaborative recollection engine 150 may also exchange data via computing devices 102a from other users 101a, at least some of whom may have a direct or indirect interpersonal relationship (e.g., as a parent, a child, a friend, an employer, an employee, a mentor, etc.) with user 101. Note, however, an interpersonal relationship need not exist between user 101 and any of other users 101a.

Further, the term “collaborative” may refer to any data representing linguistic exchanges (e.g., text-based or orally-based exchanges) between user 101 and any of other users 101a, whereby the data representing linguistic exchanges may be used to detect, evaluate, track, communicate, and/or treat a presence of a cognitive disorder associated with any of users 101 and 101a. According to some examples, collaborative recollection engine 150 may be configured to identify subsets of users 101 and 101a that may have one or more similar user attributes (e.g., a similar age range, similar sex, similar educational levels, similar languages, similar interests, similar demographics, similar occupations, similar vacation destinations, similar college experiences, etc.), and may further be configured to identify unknown (e.g., personally unknown) users 101a who may share similar recollections or memories. As cognitive abilities of a user may be a function of user attributes, one or more similar user attributes may be implemented to correlate linguistic capabilities among samples or cohorts of users to detect cognitive abilities or deviations therefrom. For example, longitudinal changes in language production due to age, regardless of whether users 101 and 101a are healthy or cognitively impaired, may be analyzed, tracked, or detected using linguistic engine 160.

To illustrate, consider that collaboratively-derived linguistic samples may be analyzed to determine a cognitive state of one or more users. For example, one of users 101a may input via computing device 102a a response 116 challenging a term, word, concept, or fact associated with originally-posted entry 112. Linguistic engine 160 may be configured to analyze response 116 to detect a potential conflict by correlating text and its subject matter between response 116 and original posted entry 112 to identify a relative degree of relevancy. For example, a common concept of “surfing competition” 119a and 119b may be detected. Also, the exchanges of text occurred within relatively short range of time during text was entered, such as a difference of 4 days. Hence, a relative degree of relevancy may be determined, for example, by computing algorithmic-based cosine similarities (or other like algorithms) to measure a degree of similarity between words, portions of sentences, and linguistic samples of any size. Linguistic engine 160 may further be configured to identify conflicting terms 118a (e.g., “Kuta Beach” and “Bali”) and 118b (e.g., “Waikiki Beach” and “Hawaii”), and monitor a response or action by user 101a to determine whether a lapse in memory recall may be an issue. In some cases, linguistic engine 160 may also be configured to monitor how frequently a term generates a conflict or is challenged. At a threshold frequency, linguistic engine 160 may detect an anomalous amount of challenges and may perform further computations to adapt a data model to enhance accuracy when determining a cognitive state. Alternatively, cue processor 152 may be configured to automatically detect a challenged word or concept (e.g., “Waikiki Beach” versus “Kuta Beach”), and generate a clarifying prompt 162 for presentation to user 101. In response, user 101 may select an input 163 to confirm Bali or select an input 164 to indicate Hawaii. If user 101 did enter the term inadvertently, user 101 may select input 165 to indicate that entry of “Kuta Beach” was just a mistake, or may select input 166 to indicate a lapse in memory.

According to various examples, collaborative recollection engine 150 and/or linguistic engine 160, as well as any constituent component therein, may be configured to receive different types of data representing linguistic samples from any number of multiple sources. For example, collaborative recollection engine 150 and/or linguistic engine 160 may be configured to receive message data 124b based on the exchange of electronic mail (e.g., email 120). Linguistic engine 160 may extract text-based linguistic samples from email 120 for analyzing patterns of language to determine a cognitive state.

As another example, collaborative recollection engine 150 and/or linguistic engine 160 may be configured to receive linguistic samples derived from short-hand or abbreviated representations of text. As shown, user 101 may implement a mobile computing device 130 including an application 132 configured to automatically transmit text-based compressed message data 124c, which may include “text messages” (e.g., SMS messages, text messaging, instant messaging (“IM”), etc.). Notably, text messaging commonly employs short form text or abbreviated representations of words or phrases, such as “LOL” to represent “laugh out loud,” which may convey a humorous interjection. Text messaging also may employ the use of “emojis” or other graphical representations of emotional states. Appropriate usage of acronyms and emojis may be correlated, at least in some cases, to changes in cognitive abilities.

In yet another example, collaborative recollection engine 150 and/or linguistic engine 160 may be configured to receive linguistic samples derived from audio data. In one implementation, application 132 may be configured to monitor one or more microphones and one or more speakers in mobile computing device 130, and may be further configured to transmit audio communication data 124d (as audio data or text converted from audio) to collaborative recollection engine 150 and/or linguistic engine 160. As such, text derived from conversational speech (e.g., in a telephone call) may be stored in a database or memory store for analysis to determine cognitive abilities. In still yet another example, application 132 may be configured to continuously (or semi-continuously under control of voice activation) monitor an ambient environment to capture speech generated by user 101 to further supplement stored data representing words, phrases, sentences, parts of speech, linguistic features, and other characteristics of language derived from ambient conversational speech.

In view of the foregoing, the structures and/or functionalities depicted in FIG. 1A as well as other portions within, illustrate one or more applications, algorithms, systems and platforms to detect, evaluate, analyze, identify, track, communicate, and/or treat a presence of a cognitive disorder, according to some embodiments. In at least one example, linguistic samples may be retrieved in the course of a user's communications (verbal or written) and analyzed in-situ. In particular, generation and analyzation of linguistic samples may be performed in-situ (e.g., without requiring clinical extraction or analyzation of text). Hence, textual analysis to predict cognitive decline may be performed non-invasively (or less invasively) in-situ than otherwise might be the case. In some examples, a determination as to whether structures of sentences may be influenced by cognitive deficiencies may be based on analyzing linguistic samples derived or received from multiple sources of linguistic samples. For example, analyzing text of memoir-style content written by users 101 and 101a via an online social engagement platform (e.g., a collaborative recollection platform and engine 150) may be correlated, and, thus, augmented by analyzing linguistic samples derived from other sources (e.g., sources of conversational speech). Specifically, accuracy in determining a presence of a cognitive deficiency may be enhanced when different sources of linguistic samples may be analyzed (simultaneously or otherwise) to detect or predict cognitive decline. Hence, a cognitive deficiency may be more likely identified as linguistic samples are tracked across a number of the multiple sources. Further, linguistic samples may be generated from any number of sources and may be tracked longitudinally by comparing language progression over time in comparison to previously recorded and analyzed linguistic samples. Should user 101 prefer usage of one source of linguistic samples (e.g., a mobile phone) relative to other sources over time, then collaborative recollection engine 150 and/or linguistic engine 160 may continue to monitor the progression of language independently or with less dependency on other sources. Additionally, a collaborative recollection platform and engine 150 and/or linguistic engine 160 may facilitate forming datasets using relatively larger sample sizes as cognitive deficiency detection is passive and may not require interactions with a professional or a clinic, at least initially. As the sample size increases, so does an accuracy of positively predicting a cognitive issue. Therefore, collaborative recollection platform and engine 150 and/or linguistic engine 160 may provide a rich set of data analyzed over a greater amount of user or people than may be the case in clinical environments.

In at least some examples, collaborative recollection engine 150, cue processor 152, and/or linguistic engine 160 may be configured to generate a prompt to capture data representing a memory, whereby the data may be analyzed to, for example, to determine one or more linguistic attributes or metrics (e.g., “Developmental Level density,” or “D-level,” and “Idea-density” or a “P-density” value, and the like). One or more linguistic attributes or metrics may include user attributes (e.g., similar age range, similar interests, similar occupations, similar vacation destinations, similar college experiences, etc.), and that may be used to identify unknown (e.g., personally unknown) users 101a and 101 to each other to share similar recollections or memories, or to provide each other with references to media content (e.g., images, text, audio, etc., whereby a “unit of content” may refer to a portion of content, such as an image, a portion or paragraph of text, a song or sound, etc.) that may unconsciously may trigger a memory by a user that otherwise might be forgotten. The one or more linguistic attributes or metrics may include location attributes, activity attributes, and the like.

As an example, collaborative recollection engine 150, cue processor 152, and/or linguistic engine 160 may be configured to generate a prompt (e.g., presented similarly as prompt 162) to capture data representing a memory design to evaluate a user's cognitive recall capabilities. In this example, a prompt prompts that may related to “flashbulb memories” (e.g., “Where were you when JFK was shot?,” or “What do you remember about the Apollo 11 moon landing?”). Such prompts that may be generate from time-to-time may be used to analyze a user's cognitive recall capabilities across a broad spectrum of users in the same age group or range who experienced the same event.

A flashbulb memory is a vivid, enduring memory associated with a personally significant and emotional event, often including such details as where the individual was or what he or she was doing at the time of the event. People often experience such memories as having similar quality of a “photograph” taken at the moment they experienced the event, and such memories may have be associated with a relatively high confidence that a flashbulb memory is accurate.

If a user has created a memory from a prompt, then collaborative recollection engine 150 can be configured to show the same prompt to the user after a specified interval has lapsed (e.g., one year, or over successive periods of time). The original response could be masked or temporarily hidden from a user when the prompt is re-displayed. Once the second memory has been recorded, the user can view both responses. In some cases, collaborative recollection engine 150 may also provide an analysis of how they differ, in terms of word choice, sentence structure and so forth.

Note that long form data need not be limited to recollections 112, and can be any text entry and any content entry for any reason, such as a post to a FaceBook® platform or any other online or cloud-based platform or system. Further, collaborative recollection platform and engine 150 and/or linguistic engine 160 may be configured to extract linguistic samples (for predicting a cognitive state) from any social network, social media, and/or social application (hereafter “social media”) such as Twitter® of San Francisco, Calif., Snapchat® as developed by Snap® of Venice, Calif., Messenger as developed by Facebook®, WhatsApp®, or Instagram® of Menlo Park, Calif., Pinterest® of San Francisco, Calif., LinkedIn® of Mountain View, Calif., and others, without limitation or restriction.

The structures and/or functions of any of the above-described features can be implemented in software, hardware, firmware, circuitry, or a combination thereof. Note that the structures and constituent elements described above (or herein), as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. In some examples, the described techniques may be implemented as a computer program or application (hereafter “applications”) or as a plug-in, module, or sub-component of another application. The described techniques may be implemented as software, hardware, firmware, circuitry, or a combination thereof. If implemented as software, the described techniques may be implemented using various types of programming, development, scripting, or formatting languages, frameworks, syntax, applications, protocols, objects, or techniques, including Python™, ASP, ASP.net, .Net framework, Ruby, Ruby on Rails, C, Objective C, C++, C#, Adobe® Integrated Runtime™ (Adobe® AIR™), ActionScript™, Flex™, Lingo™ Java™, JSON, Javascript™, Ajax, Perl, COBOL, Fortran, ADA, XML, MXML, HTML, DHTML, XHTML, HTTP, XMPP, PHP, and others, including SQL™ SPARQL™ Turtle™, etc., which may be configured to access databases, data structures, and the like, including data models or APIs. The described techniques may be varied and are not limited to the embodiments, examples or descriptions provided.

FIG. 1B is a diagram depicting an example of an application stack configured to implement applications to predict a cognitive state based on text-based analysis, such as depicted in user interface 110 of FIG. 1A, according to some examples. A network protocol 171, such as HTTP (or the like), may be layered upon a network layer 173, which may include IP-based networks and protocols or any other type of network. Further, application stack 151 is shown to include a JavaScript engine 157, a document object model (“DOM”) 161, and a cascading style sheet (“CSS”) language 167, as an example. A subset of application 151 may include executable instructions to implement a cue processor 149, and another subset of application 151 may include executable instructions to implement a linguistic engine 153. One or more components of application stack 151 may be implemented within, or distributed over, one or more computing devices (e.g., servers) configured to implement collaborative recollection engine 150 of FIG. 1A, as well as over one or more computing devices 102 and 102a (e.g., including browser applications and/or clients).

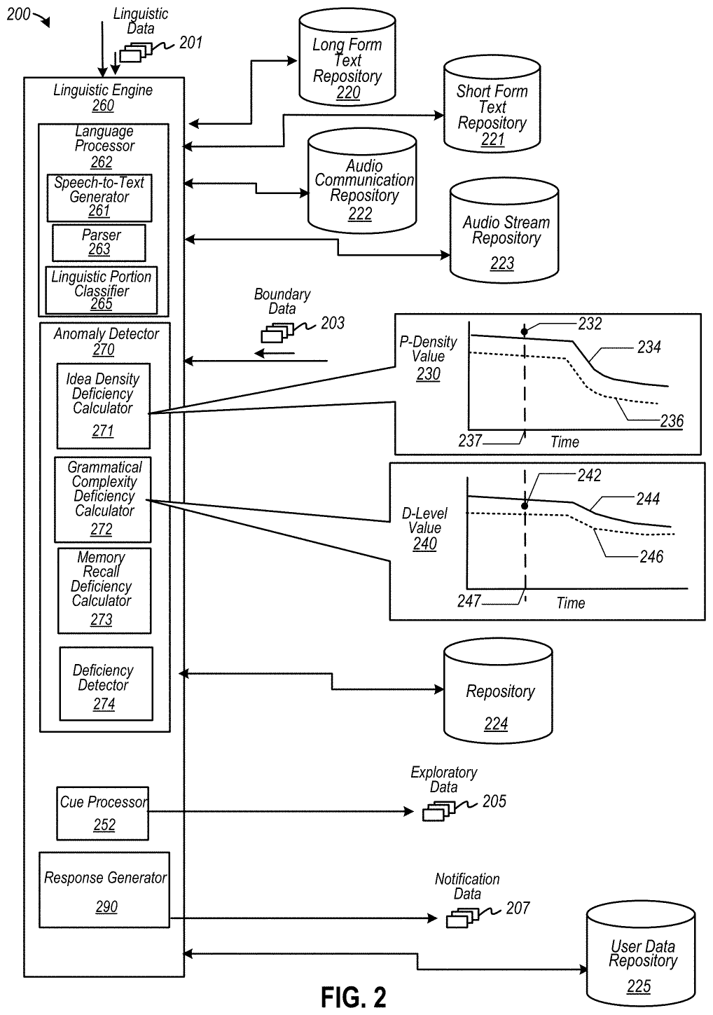

FIG. 2 is a diagram depicting an example of a linguistic engine configured to predict, detect, evaluate, track and/or identify a likelihood of a presence of a cognitive disorder, according to some examples. Diagram 200 depicts a linguistic engine 260 including a language processor 262, an anomaly detector 270, a cue processor 252, and a response generator 290, according to some examples. One or more elements depicted in diagram 200 of FIG. 2 may include structures and/or functions as similarly-named or similarly-numbered elements depicted in other drawings, or as otherwise described herein, in accordance with one or more examples. Also, one or more constituent components of linguistic engine 260 may be configured to implement one or more functionalities and/or structures set forth in one or more examples described in U.S. patent application Ser. No. 15/961,432, which is incorporated herein by reference.

Further, diagram 200 depicts that language processor 262 may be configured to receive linguistic data 201, which may include text-based data, audio-based data, language-specific data (e.g., text in the French language), and the like, and may be further configured to process linguistic data 201 from any source of linguistic samples. Linguistic data 201 may include alpha-numeric text that forms words, clauses, or phrases that constitute one or more sentences (or portions thereof), and may originate from any of a number of sources of alpha-numeric text, according to some examples. For instance, linguistic data 201 may include one or more of data 124a, 124b, 124c, 124d, and 124e of

FIG. 1A.

Referring back to FIG. 2, language processor 262 may include a speech-to-text generator 261, a parser 263, and a linguistic portion classifier 265. Speech-to-text generator 261 may be configured to convert portions of audio data into units of text, whereas the audio data may be generated responsive to a user speaking during a conversation, which may transpire via a mobile computing device or phone. Parser 263 may be configured to perform natural language processing (“NLP”) to identify and form words, clauses, and sentences (and portions thereof) in view of lexical, syntactic and semantic rules or instructions. In some examples, parser 263 may perform “parts-of-speech” identification and tagging of words in accordance with a classification assigned to a unit of language in accordance with a syntactic function (e.g., a grammatical relationship between one constituent unit of language to another within a syntactic construction). Syntactic function categories to which a word may be assigned include at least one or more of a noun, pronoun, adjective, verb, adverb, determiner, preposition, conjunction, interjection, and the like.

Linguistic portion classifier 265 may be configured to classify one or more portions of the sentence in preparation for additional processing, including calculation of one or more linguistic measures or parameters to apply to anomaly detector 270. In some cases, linguistic portion classifier 265 may be configured to perform enhanced “parts-of-speech” processing to further identify and tag one or more words as a “proposition,” whereby a proposition may include a verb (e.g., a main verb) and one or more of a subject, object, indirect object, and the like to express a unit of understanding or an idea. In some examples, linguistic portion classifier 265 may be configured to identify portions or segments of sentences as either “right-branching” or “left-branching,” whereby left-branching is more indicative of linguistic complexity. As persons age, they tend to prefer right-branching sentence constructions, which are simpler. Right-branching sentences include an embedded clause (e.g., verbs, objects, modifiers, etc.) disposed to the right of a main clause (e.g., subject), whereas the embedded clause may be disposed prior to (e.g., to the left of) the main clause in left-branching sentence constructions. Linguistic portion classifier 265 may also be configured to perform other classifications that may also be performed by an anomaly detector 270 and/or its constituent components.

According to some implementations, language processor 262 and its constituent components may be configured to identify sources of text, perform parsing, and classify linguistic portions. Further, language processor 262 may be configured to store data representing linguistic samples and corresponding metadata (e.g., tagged parts-of-speech, classifications, etc.) in repositories 220, 221, 222, and 223. In long form text repository 220, linguistic data derived from memoir-style data entries (e.g., recollection data of FIG. 1A), emails, and the like may be stored as a dataset or “corpus” of words, clauses, sentence segments, sentences, and collections of sentences derived by data generated by a particular user (or subset of users) using long-form text entry techniques. In short form text repository 221, linguistic data derived from compressed message data (e.g., data 124c of FIG. 1A, including acronyms, emojis, etc.), may be stored as another “corpus” of text derived by data generated by a particular user (or subset of users) using short-form text entry techniques, such as SMS text protocols. In audio communication repository 222, linguistic data derived from audio data (e.g., data 124d of FIG. 1A, including spoken language), may be stored as a “corpus” of text derived by data originating from one or more users (or subset of users) engaged in oral conversations in which data representing a challenge to a word, concept, or fact may be captured and stored in repository 222. In audio stream repository 223, linguistic data derived from audio data (e.g., data 124e of FIG. 1A, including ambient spoken language), may be stored as a “corpus” of text derived by data originating from a user (or subset of users) in which oral speech may be captured passively.

In accordance with at least one example, a data model may specify that each unit of language (e.g., word, etc.) may be associated with tag that may link a unit of language to: (1.) data representing one or more users (e.g., one or more user identifiers), (2.) data representing one or more ranges of time (e.g., date of data entry or storage in repositories 220 to 223), (3.) data representing one or more locations, (4.) data representing one or more activities, or (5.) data representing any other data object. Therefore, in view of the foregoing, a unit of language may include time-related tag data, location-related tag data, and activity-related tag data, as well as user identifier and/or user attribute tag data (e.g., user-related attribute tag data may include age-related tag data, gender-related tag data, education level-related tag data, etc.). Linguistic engine 260 may use the aforementioned tag data to correlate one linguistic sample with another linguistic sample, such as relating the text of recollection of 112 to the text of a responding comment 116 of FIG. 1A, whereby a correlation may be determined based on a degree of relevancy (e.g., using cosine similarities). Further, linguistic engine 260 may be configured to identify similar or equivalent units of language over any number of repositories 220 to 223 so as to correlate units of language and resultant influences in language production that may facilitate detection of cognitive deficiencies. Linguistic engine 260 may also use sentiment analysis of a linguistic sample to determine, for example, a degree of negativity embodied in text of a responding comment 116 of FIG. 1A, whereby the negativity may indicate a conflict or disagreement of a fact or word. Such a conflict may reveal a deficiency in memory recall. In at least one embodiment, the above data model specifying various tag data with a unit of language may be implemented similar or equivalent to an example of a cue data model described in U.S. patent application Ser. No. 15/961,432, which is incorporated herein by reference.

Diagram 200 depicts anomaly detector 270 including an idea density deficiency calculator 271, a grammatical complexity deficiency calculator 272, a memory recall deficiency calculator 273, and a deficiency detector 274 in accordance with some examples. Anomaly detector 270 may be configured to calculate one or more linguistic measures for one or more linguistic samples, which may include data representing a value indicative of, or correlatable to, a cognitive state that may be compared to boundary or threshold data associated with cognitive deficiencies. Hence, anomaly detector 270 may be configured to determine whether the cognitive ability of a user is within a “normal” (or healthy) range of values or within an “anomalous” (cognitively impaired) range of values. An “anomalous” (cognitively impaired) range of values may indicate a presence of one or more cognitive states resulting from the effects of Alzheimer's disease, chronic traumatic encephalopathy (“CTE”), age-related cognitive decline, or the like.

Idea density deficiency calculator 271 may be configured to compute a linguistic measure, such as an “Idea-density” or a “P-density” value 230 relative to differences in time (e.g., age). In some examples, P-density may refer to “propositional density,” and idea density deficiency calculator 271 may be configured to measure “propositional content,” which may be a rate at which ideas or a proposition may be expressed as a function of a main verb relative to it arguments or other units of language (e.g., a subject, an object, an indirect object, or the like). In other examples, idea density deficiency calculator 271 may be configured to calculate P-density value 230 as a number of expressed propositions (e.g., prepositions, verbs, adverbs, adjectives, and conjunctions) divided by a number of words in a unit of a linguistic sample (e.g., a sentence, a group of sentences, or a number of words, such as 10 words or 100 words). As lower values of P-density may correlate to an increased risk of developing Alzheimer's disease, idea density deficiency calculator 271 may be configured to evaluate a likelihood that a user is healthy or may be experiencing onset of a cognitive impairment.

In the example shown, a boundary or threshold data value 236 indicative of a cognitive deficiency may be provided as boundary data 203 (data based on clinical studies, or data derived empirically by linguistic engine 260). Boundary data 203 may include data representing aggregated P-density values 236 over different ages, whereby the aggregated P-density values 236 may have been derived from persons being diagnosed with, for example, Alzheimer's disease or a propensity to develop Alzheimer's disease based on a genetic condition. Further, P-density values 234 may include data representing linguistic measures calculated based on linguistic samples generated by individuals not symptomatic of having a cognitive impairment (e.g., healthy individuals). Diagram 200 depicts a user's P-density value 232 at a point of time (or age) 237 relative to, for example, at least one linguistic boundary value 236.

Grammatical complexity deficiency calculator 272 may be configured to compute a linguistic measure, such as a “Developmental Level density,” or a “D-level” value 240 relative to differences in time (e.g., age). In some examples, grammatical complexity deficiency calculator 272 may be configured to measure “sentence complexity,” which may be a function of an amount of embedding and a type of embedding used to create complex sentences. Also, D-Level values 240 may be influenced by a speaker's or writer's working memory. In some cases, capabilities of working memory may decrease with age, thereby increasing the burden upon a user to form more complex sentences. D-Level value 240 may be computed by classifying a unit of language, such as a sentence, as one of a number of categorized levels (e.g., from either “level 0” or “level 1” to “level 7”), and by assigning a value equivalent to a corresponding D-Level category. In other examples, grammatical complexity deficiency calculator 272 may be configured to calculate D-Level value 240 as “0” points if a sentence is analyzed and determined to be a simple, one-clause sentence, whereas “1” point may be assigned to a sentence categorized as including an infinitival complement sharing a common subject as a main clause, which may be associated with “level 1.” Grammatical complexity deficiency calculator 272 may be configured to calculate D-Level value 240 as “2” points if two noun phrases are joined by a conjunction, which defines level 2. As sentence complexity increases, D-Level value 240 increases up through level 7 in which multiple kinds of embedding within a sentence yields “7” points as D-Level value 240. In other examples, grammatical complexity deficiency calculator 272 may be configured to calculate D-Level values 240 for each sentence in a linguistic sample, and may further be configured to generate an average D-Level value 240 for the linguistic sample. In the example shown, a boundary or threshold data value 246 indicative of a cognitive deficiency may be provided to grammatical complexity deficiency calculator 272 via boundary data 203.

Boundary data 203 may include data representing aggregated D-Level values 246 over different ages, whereby the aggregated D-Level values 246 may have been derived from persons being diagnosed with, for example, Alzheimer's disease. Further, D-Level values 244 may include data representing linguistic measures calculated based on linguistic samples generated by individuals not symptomatic of having a cognitive impairment (e.g., healthy individuals). As shown, diagram 200 depicts a user's D-Level value 242 at a point of time (or age) 247 relative to, for example, at least one linguistic boundary value 246.

Memory recall deficiency calculator 273 may be configured to monitor and detect possible inconsistencies and usage of terms by a user that may be influenced by a cognitive deficiency that manifests as a lack of memory recall or diminished working memory. In some examples, memory recall deficiency calculator 273 may identify a unit of language (e.g., a concept, a word, etc.) that may be challenged by another user during an exchange of communications. In some instances, the other user challenging a unit of language may proffer an alternative term in a form of a question. Resolution of a conflicted term may include analyzing data representing an indication specifying that the inconsistency is either a simple mistake or due to a lapse in memory. Memory recall deficiency calculator 273 may be configured to track numbers of challenged terms per duration of time, and if a threshold amount of challenges occur in a specific interval of time, memory recall deficiency calculator 273 may be configured to generate a value associated with a likelihood that a user's ability to recall a memory is affected. In one example, memory recall deficiency calculator 273 may invoke cue processor 252 to regenerate exploratory data 205 that is configured to cause presentation of selectable inputs in a user interface associated with a user. Each selectable input may be associated with a condition of a user to confirm whether the user has experienced any head trauma (e.g., diagnosed with concussions), played any full-contact sport (e.g., rugby or American football), and whether any cognitive disease or impairment is experienced by any genetic family member. A value of memory recall deficiency may be modified or weighted as a function of a particular selected input (e.g., “yes,” I played football and suffered 3 concussions). As such, a data model implemented by linguistic engine 260 may implement an adjustable or adaptive data model based on modifiable weighting factors associated with memory recall or any other cognitive state, linguistic measure value, or functionality described herein.

Deficiency detector 274 may be configured to receive data representing values of linguistic measures calculated by idea density deficiency calculator 271, grammatical complexity deficiency calculator 272, and memory recall deficiency calculator 273. Deficiency detector 274 may be further configured to identify one or more weighting factors to be applied to a linguistic measure value, such as one or more of a P-density value, a D-Level value, and a memory recall deficiency value.

In some examples, deficiency detector 274 may be configured to implement an adaptive data model that may be configured to modify computations as a function of modified or recalibrated weighting factor values. For example, a weighting factor value may be adapted as a function, for example, of a user's age, sex, education level, or any other user attribute, or any other attribute or characteristic associated with any number of linguistic samples. User attributes and other attributes may be stored in, and retrieved from, user data repository 225. In at least one implementation, deficiency detector 274 may normalize (e.g., set values within a range from 0 to 1) weighted values of a P-density value, a D-Level value, and a memory recall deficiency value, whereby a combination of the weighted values may be aggregated to form a composite value representative of cognitive state. The weighted values of linguistic measures and a composite value may be stored in repository 224 for purposes of comparing subsequently derived values to characterize a rate of cognitive declination, if detectable, as a user ages. Deficiency detector 274 may, at least in some cases, identify a type of cognitive disorder as a function of the aforementioned computed linguistic measure values. Response generator 290 may be configured to generate a notification data 207 that may indicate that a cognitive deficiency or anomaly may be present. Notification data 207 may describe a user's cognitive state or abilities and may be transmitted to a medical professional or family member.

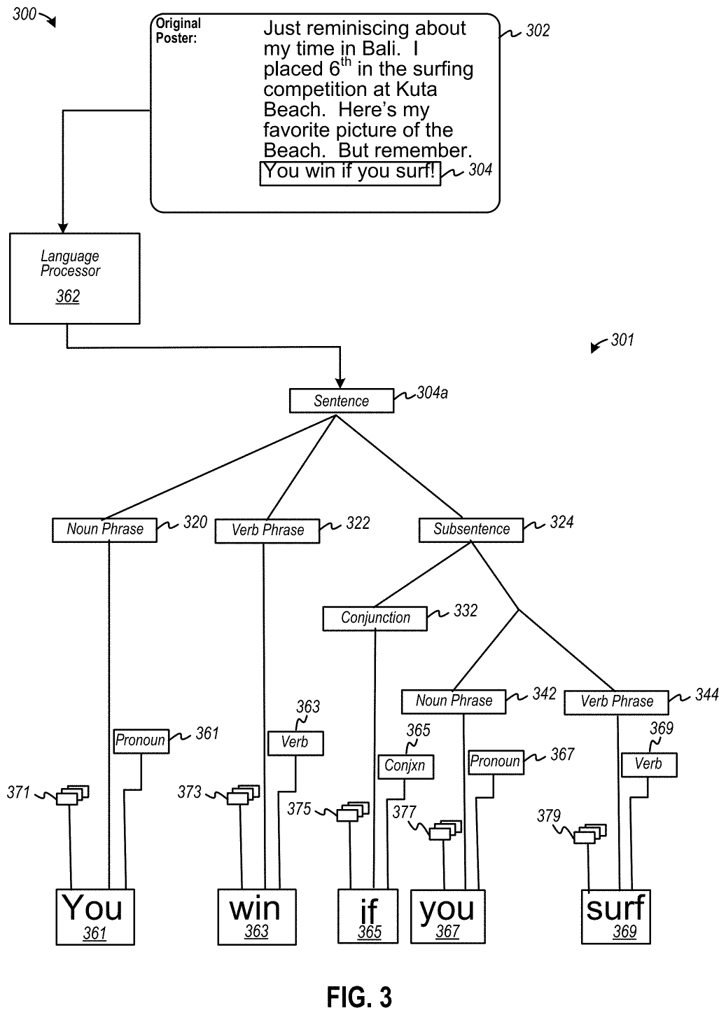

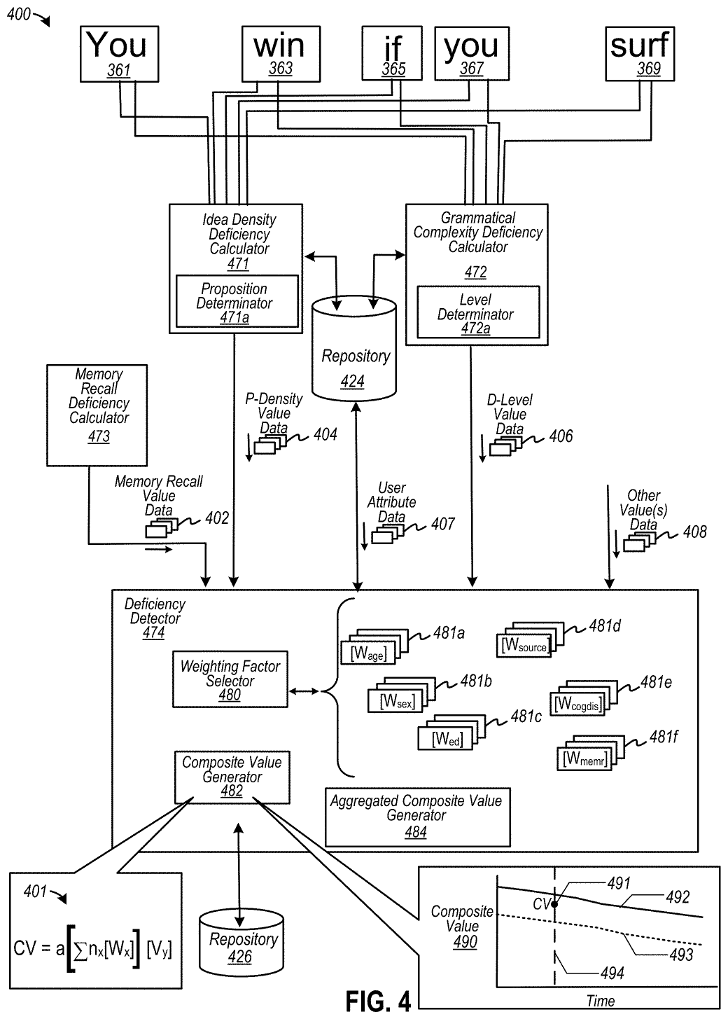

FIGS. 3 and 4 are functional block diagrams depicting examples of the functionalities of a linguistic engine, according to at least one embodiment. Diagram 300 depicts an example operation of a language processor 362 that may be configured to receive a linguistic sample 302 including a portion constituting a sentence 304. Language processor 362 may include a speech-to-text generator, a parser, and a linguistic portion classifier, none of which is shown. Language processor 362 also may parse through characters (e.g., alphanumeric characters), which may be tokenized, to identify units of language, such as words. For example, sentence 304 may include units of language 361, 363, 365, 367, and 369 that may constitute words “You,” “win,” “if,” “you,” and “surf,” respectively.

Further to diagram 300, language processor 362 may be configured to analyze sentence 304 to determine a hierarchal syntactic structure 301 of a string, such as sentence 304, and interrelationships among units of language 361, 363, 365, 367, and 369. As shown, language processor 362 may generate relationships or links in a form of a parse tree, such that sentence 304 may be associated with a root node 304a, which includes nodes 320, 322, and 324 representing a noun phrase 320, a verb phrase 322, and a subsentence 324, respectively. Further, language processor 362 may generate relationships or links in a form of a sub-parse tree based on node 324, which includes nodes 332, 342, and 344 that represent a conjunction, a noun phrase, and a verb phrase, respectively.

Language processor 362 may also be configured to tag words 361, 361, 363, 365, 367, and 369 with data representing a “pronoun,” a “verb,” a “conjunction,” a “pronoun,” and a “verb,” respectively, all of which may define a part-of-speech. As linguistic sample 302 is extracted from a text-based digital medium in long form (e.g., text entered online as a recollection of FIG. 1A), syntactic structure 301 and words 361, 363, 365, 367, and 369 may be stored in a long form text repository (e.g., repository 220 of FIG. 2). Therefore, metadata describing a source of text may be linked or tagged to each word 361, 363, 365, 367, and 369 in tag data 371, 373, 375, 377, and 379, respectively. Metadata describing similar or equivalent words, as well as synonyms, disposed in other repositories (e.g., repositories 221 to 223 of FIG. 2) may be linked or tagged to each word as in tag data 371, 373, 375, 377, and 379. In some cases, each word 361, 363, 365, 367, and 369 may be tagged with data 371, 373, 375, 377, and 379, respectively, to indicate a frequency or a number of times that a word has been a subject of a challenge-response exchange, which may be indicative of a user's difficulty in recalling a particular word from memory.

Tag data 371, 373, 375, 377, and 379 may include user attribute data (e.g., specifying similar age range, similar sex, similar educational levels, similar languages, similar interests, similar demographics, similar occupations, similar vacation destinations, similar college experiences, etc.). Tag data 371, 373, 375, 377, and 379 may include data representing a location (e.g., a geographic location, an institution or building, a domicile, etc.), data representing an activity (e.g., surfing, swimming, working, etc.), and other types of data to facilitate correlations between units of language or linguistic samples, whereby correlatable words (e.g., via cosine similarities) may be used to link linguistic samples originating from various sources to a particular user or individual. As such, correlatable linguistic samples may be monitored and tracked over time as a function of at least a user's age to evaluate cognitive abilities over time.

FIG. 4 includes a diagram 400 depicting other example operations of a language processor that may be configured to further process units of language 361, 363, 365, 367, and 369 constituting words “You,” “win,” “if,” “you,” and “surf,” respectively. Hence, a language processor may be configured to evaluate linguistic samples to predict whether language production may be influenced by a cognitive disorder or deficiency, thereby establishing that a user may be afflicted with a cognitive impairment. Units of language 361, 363, 365, 367, and 369, as a sentence, may be inputted into any number of linguistic measure calculators, such as idea density deficiency calculator 471 and grammatical complexity deficiency calculator 472, which may to generate P-density value data 404 and D-Level value data 406, respectively. Also, memory recall deficiency calculator 473 may be configured to generate memory recall value data 402 as a linguistic measure value for determining a cognitive state. One or more elements depicted in diagram 400 of FIG. 4, such as idea density deficiency calculator 471, grammatical complexity deficiency calculator 472, and memory recall deficiency calculator 473, may include structures and/or functions as similarly-named or similarly-numbered elements depicted in other drawings, or as otherwise described herein, in accordance with one or more examples.

According to various embodiments, any number of linguistic measure calculators may be implemented to generate any number of linguistic measure values that may be inputted as other value(s) data 408 into a deficiency detector 474. For example, a calculator to determine “Brunet's W index” may be implemented to determine lexical diversity and richness of a speaker's or writer's vocabulary, whereby “W” is a function of a total number of words used, N, and a vocabulary size, V. Also, a calculator may be configured to implement “Honore's R Statistic” to compute lexical richness as a function of a total number of words used, N, a vocabulary size, V, and a number of words mentioned only once, V1. In some cases, a value indicative of a “level of specificity” may be computed to convey an amount of detail expressed in a linguistic sample, and a value representing an average sentence length may be computed as well. Moreover, a computation to determine value representing a Mean length of utterance (“MLU”) may be performed to indicate a level of language proficiency. Any of the aforementioned values may be provided as data 408 to deficiency detector 474 to evaluate a cognitive state.

In some examples, deficiency detector 474 may be implemented as a probabilistic prediction processor, whereby the adaptive prediction model may be trained on text-based linguistic samples text extracted from a sample of people with Alzheimer's disease (or other cognitive dysfunctions) and a sample of people that include healthy, older adults. As such, deficiency detector 474 may be configured to probabilistically analyze linguistic measure values, and compare and/or correlate those values to linguistic measure values of healthy adults and to those suffering from cognitive dysfunction (e.g., as diagnosed). Upon detecting a linguistic performance level outside of a certain threshold (e.g., probabilistically assigned by a model), deficiency detector 474 may be configured to cause generation of an electronic message as a notification that a linguistic parameter or measure value associated with an individual's writing or transcribed speech is not within a boundary for healthy cognitive levels for individuals of, for example, a particular age group. Hence, methods, algorithms, and models described herein may be configured to determine correlations in changes in writing ability that may be indicative of cognitive decline, at least in some examples.

Further to the example shown in FIG. 4, repository 424 may be configured to receive and store data representing P-Density value data 404, D-Level value data 406, as well as user attribute data 407, which may include data representing an age (or age group) of a user, data representing a gender of a user, data representing an educational level of a user, and any other attribute of a user that may be considered when evaluating a cognitive state.

In some examples, idea density deficiency calculator 471 may include logic (e.g., hardware and/or software) implemented as a proposition determinator 471a that may be configured to generate P-density value data 404. As one example, a P-density value may be computed as an average number of ideas (i.e., propositions) expressed per unit of text, whereby a unit of text may be set as “10 words.” To illustrate, consider the following sentence: “Skinny Jack Spratt came from the moon.” Proposition determinator 471a may be configured to detect “7 words,” and the following three (3) ideas: (Idea 1.) “Jack Spratt”==“came,” (Idea 2.) “Jack Spratt”==“came from the moon,” and (Idea 3.) “Jack Spratt”==“Skinny.” Hence, a value of P-density may be computed as [10/7]*3=4.29. In some cases, a value of may be determined as an average value over each word and sentence of a linguistic sample.

Grammatical complexity deficiency calculator 472 may include logic (e.g., hardware and/or software) implemented as a level determinator 472a that may be configured to generate D-Level value data 406. As one example, a D-Level value may be computed as a number associated with a D-Level classification, which may range from a value of “0” to about “7” or “8.” To illustrate, consider the sentence “You win if you surf” is analyzed and computed to match a lexical and syntactical pattern matching a “Level 6” classification in a Developmental Level scale. At level 6, sentences may be joined by a subordinating conjunction, whereby a first sentence “You win,” which may be joined to a second sentence “you surf” via a subordinating conjunction “if.” Thus, a value of D-density is “6,” which may be transmitted as level value data 406. In some cases, a value of D-Level may be determined as an average D-Level value over each sentence of a linguistic sample.

In the example shown, deficiency detector 474 may be configured to analyze one or more linguistic measure values, each of which may be modified or adjusted by one or more weighting factors of an adaptive data model, to detect whether one or more linguistic samples over time exhibit characteristics that may fall outside the range of values associated with healthy or normal cognitive abilities. Here, deficiency detector 474 includes a weighting factor selector 480 and a composite value generator 482. According to some examples, weighting factor selector 480 may be configured to select or adapt specific weighting values based on relative attributes associated with a user and its linguistic sample, such as an age, a sex, an education level, a source of the linguistic sample, an indication whether a user may be diagnosed with (or is at risk of) a cognitive disorder, and a value indicative of a user's ability to recall memory, among others. Based on the above-described attributes, weighting factor selector 480 may be configured to select or adapt one or more of (1.) a corresponding weighting value based on age (“Wage”) 481a, (2.) a corresponding weighting value based on sex (“Wsex”) 481b, (3.) a corresponding weighting value based on level of education (“Wed”) 481c, (4.) a corresponding weighting value based on a source of the linguistic sample (“Wsource”) 481d, (5.) a corresponding weighting value to reflect whether a user is “at risk” of a future cognitive disorder or has been diagnosed with a cognitive disorder

(“Wcogdis”) 481e, (6.) a corresponding weighting value indicative of a capability to recall memory (“Wmemr”) 481f, or any (7.) other weighting factor value for any other attribute (not shown).

Composite value generator 482 may be configured to adjust, modify, or recalibrate a value of a linguistic measure as a function of one or more attributes (e.g., associated with a user) to form a composite value that may be compared to boundary or threshold values associated with values indicative of either normal or anomalous cognitive abilities. In some examples, composite value generator 482 may be configured to generate a composite P-density value, a composite D-Level value, or any other composite value for a linguistic measure based on, for example, one or more weighting factor values. In various examples, composite value generator 482 may be configured to generate a composite value (“CV”) 491 for at least one linguistic measure adjusted by one or more weighting factor values. Deficiency detector 474 may be configured to analyze composite value 491 at time 494 (e.g., at a user's particular age) relative to a range of values 492 indicative to relatively normal or healthy cognitive abilities, and further configured to analyze composite value 491 relative to a range of values 493 associated with relatively anomalous cognitive abilities (e.g., indicating onset of a cognitive disorder, such as Alzheimer's disease). A specific composite value (“CV”) 491 at time 494 may be stored in repository 426 for subsequent analysis and evaluation to detect potential degradation in cognitive ability at a later time.

A composite value for any linguistic measure made to be determined in accordance with any computation of the linguistic measure that may be tuned or adapted in accordance with one or more weighting factor values to, for example, adjust for one or more of relevant attributes of a user, types of sources from which a linguistic sample is received, indications of a pre-existing cognitive disorder, indications of lapses in memory recall abilities, or any other factor. In one example, a composite value may be computed in accordance with relationship 401 in which a value (“Vy”) of a linguistic measure (e.g., P-density value) may be adjusted by a value represented by an aggregation (e.g., a summation) of each of a subset of weighting factor values (“Wx”). Each weighting factor value (“Wx”) may be optionally normalized by application of a normalization factor or function (“nx”), according to some examples. A correction factor “a” may be applied to resolve errors or to fine-tune the result. Values of each of the weighting factors “Wx” may be values associated with one or more weighting values 481a to 481f.

In view of the foregoing, a weighting factor may have a value as a function of a type of source of a linguistic sample (e.g., long form text, short form text, audio communication data, etc.). A corresponding weighting factor value (e.g., Wsource 481d) for a specific source facilitates adjustment of a value of a linguistic measure. Adjustment or adaptation of a value of a linguistic measure may enhance accuracy relative to, for example, a degree of reliability that a linguistic sample from a particular source provides sufficient information from which to evaluate a cognitive state. Use of multiple sources of linguistic samples ensures comprehensive monitoring and detection of cognitive states passively, and in view of a user's behavior in preferring to use one source over another source.

Further, a value of a linguistic measure may be adjusted to compensate for data indicating a likelihood that a user's memory recall ability may be degrading, which, in turn, may assist in evaluating whether a user may be afflicted with a cognitive impairment. Consider that a memory recall value in data 402 represents a degree of likely degradation in memory recall for a user. Since users may spend less time ensuring accuracy of short form textual communications (i.e., text messages), such users likely may make more mistakes due to speed rather than in lapse in memory. Thus, reliability of a value of memory recall in association with exchanging text messages may be less than a value associated with exchanging emails or other long form based on, for example, a memoir-style online data entry medium, such as provided by CueBack Technology, Inc.

Correspondingly, a value of a weighting factor associated with a memory recall value may vary based on a type of a linguistic measure or a type of source from which a linguistic sample may be extracted.

Moreover, a value of a linguistic measure may be adjusted to compensate for data indicating a likelihood that a user may be at risk of having cognitive deficiencies due to past experiences (e.g., playing football, drinking to excess, etc.), or due to genetic familial relationships. Also, a linguistic measure may be adjusted to compensate for any confirmed diagnosis of a cognitive impairment. Thus, a weighting factor value 481e may be formed to adjust a value of a linguistic measure (e.g., P-density) to enhance accuracy and precision of determining whether a cognitive disorder is present based on a user's language production.

In at least one example, deficiency detector 474 may include an aggregated composite value generator 484 configured to blend or aggregate composite values of multiple linguistic measures, such as combining a composite value for P-density and a composite value for D-Level, in view of the functionalities and structures described herein.

According to various other embodiments, any number of other linguistic measures may be implemented to determine a cognitive state or a deficiency thereof. For example, an attribute of user describing a user as an alcoholic (or otherwise consuming large amounts of alcohol, or misuse of drugs) may be evaluated as increased alcohol consumption may correlate to an increased likelihood of cognitive dysfunction. Also, an attribute of a user describing a user as experiencing a head injury or participation in a full-contact sport (e.g., as American football or head-butting moves in soccer) may correlate to an increased likelihood of cognitive dysfunction. Further, confirmation of a cognitive-related diagnosis may also correlate to an increased likelihood that a cognitive dysfunction may be detected via analyzation of linguistic samples. Data representing the aforementioned user attributes may be provided to deficiency detector 474 as user attribute data 407.

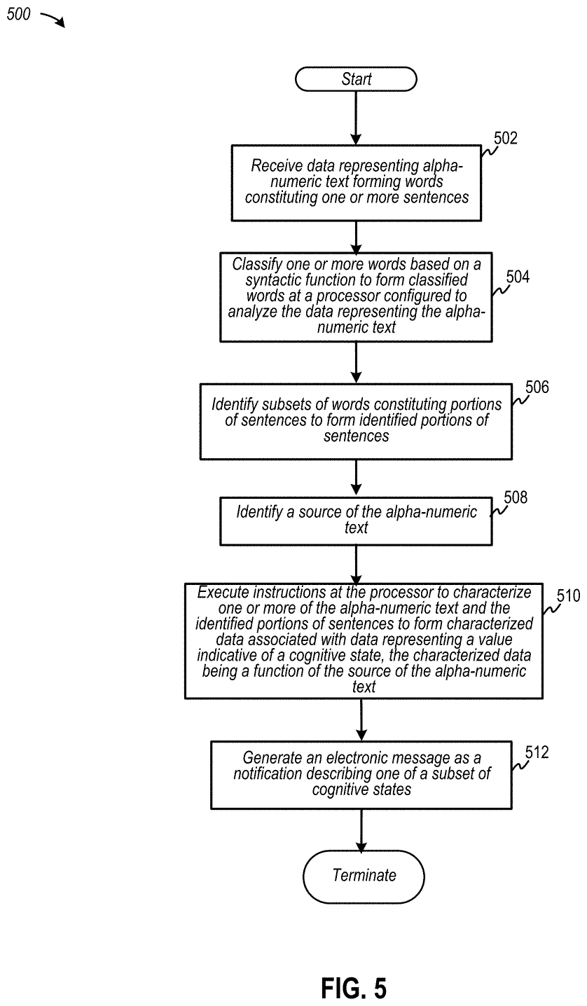

FIG. 5 is a diagram depicting a flow diagram as an example of analyzing linguistic samples to predict whether a cognitive deficiency may affect formation of a linguistic structure associated with language production, according to some embodiments. Flow 500 begins at 502, which describes that data representing alphanumeric text may be received, whereby the alphanumeric text may form units of language or words constituting one or more sentences (or portions thereof). At 504, one or more units of language or words based on a syntactic function may each be classified at a processor. A syntactic function category to which a word may be assigned include at least one or more of a noun, pronoun, adjective, verb, adverb, determiner, preposition, conjunction, interjection, and the like, or any other classification as a “part-of-speech.” The processor may be configured to form classified units of language or words to analyze data representing alphanumeric text of one or more linguistic samples and may associate (or tag) each unit of language or word with metadata configured to, for example, correlate different linguistic samples.

At 506, subsets of words for units of language constituting portions of sentences may be identified form identify portions of sentences. One or more words or subsets of words may be identified as classified to form subsequent calculations of values of linguistic measures. For example, one or more words may be identified and labeled as a “proposition,” which may relate a verb to one or more of a subject, object, indirect object to facilitate subsequent linguistic measure value computation (e.g., determining a P-density value). As another example, portions or segments of sentences may be analyzed and identified as either “right-branching” or “left-branching,” which may be determined to identify subsequently a D-Level value for a sentence. Also, clauses may be identified and analyzed to determine whether a clause is embedded as a subject of a main verb, with which to identify a sentences as having a D-Level value of “6.” Other portions or segments of sentences may be analyzed and identified to facilitate determining any linguistic measure value.

At 508, a source of the alphanumeric text may be identified. For example, the source of a linguistic sample or long form text may be an online data entry platform configured to maintain and supplement recollections, or an email. Sources of short form text include data representing text messages including abbreviations and graphical representations (e.g., emojis). Sources of text may originate as audio data representing voiced conversations or passively-captured user speech using an application on a mobile computing device to activate its microphone.

At 510, instructions may be executed at a processor to characterize one or more subsets of alpha-numeric text and identified portions of sentences to form characterized data. The characterized data may be associated with data representing a value indicative of a cognitive state, the value being indicative of a cognitive state, such as Alzheimer's disease, chronic traumatic encephalopathy (“CTE”), and age-related cognitive decline. According to some examples, characterized data formed to specify a cognitive state may be generated as a function of the source of an alpha-numeric text.

In one example, characterized data may be formed by computing a linguistic measure to form a value indicative of the cognitive state. Data representing a proposition density value (e.g., “P-density value”) per unit of text may be calculated. In some cases, a proposition density value may be indicative of a rate of ideas or propositions expressed per unit of text. Further, a proposition density value may be correlated, as a value indicative of a cognitive state, to a subset of proposition density values that may constitute a boundary or threshold of values to determine a cognitive state. Thus, analyzing and computing linguistic measure values may indicate no cognitive impairment—that is, a patient presents as healthy or with non-afflicted language production. Otherwise, a cognitive disorder may exist. In some cases, a type of cognitive disorder may be identified based a calculated cognitive state.

In another example, characterized data may be formed by computing a linguistic measure to form a value indicative of the cognitive state. Data representing a data representing a developmental level (“D-Level”) value per unit of text may be calculated. In some cases, a developmental level value may be calculated to be indicative of grammatical complexity per unit of text. Further, a D-Level value may be correlated, as a value that may constitute a boundary or threshold of values indicative of a cognitive state. Thus, analyzing and computing linguistic measure values may yield an indication of no cognitive impairment—that is, a patient presents as healthy or with non-affected language production. Otherwise, a cognitive disorder may exist. In some cases, a type of cognitive disorder may be identified based a calculated cognitive state

In yet another example, data representing a subset of alpha-numeric text may be received as a challenge response that may specify one or more words at conflict. A challenge response may be indicative of an issue of memory recall. In some examples, a challenge term and an alternative term may be identified as being at conflict. A challenge term may be a word, a phrase, a symbol conveying a meaning, or the like that may have been originally communicated, whereas an alternative term may be provided as a suggested replacement to reconcile consistencies of terms. Response to an apparent conflict, a value associated corresponding by an amount indicative of the issue of memory recall may be modified to form a memory recall deficiency value. In some cases, a portion of a user interface may be presented to include a user input to confirm whether a challenge term ought to be replaced by an alternative term.

In forming the above-described characterized data by calculating a P-density value, a D-Level value, and/or a memory recall deficiency value, one or more of the aforementioned values may be modified within application of one or more weighting factors. In some example, one or more weighting factors may be available for use, each weighting factor corresponding to one of a multiple sources of alpha-numeric text. As such, if a linguistic sample is used to compute linguistic measure values (e.g., calculating a P-density value, a D-Level value, and/or a memory recall deficiency value), a weighting factor associated with a specific source of text, such as audio data captured from a cellular phone call, may be used to tune one or more values, such as composite values, that may specify whether a cognitive deficiency may be present.

At 512, an electronic message may be generated as a notification to describe one of a subset of cognitive states. The electronic message may be transmitted to any user account or person who may have an interest, including, but not limited to, immediate family members or loved ones, as well as medical professionals and doctors.

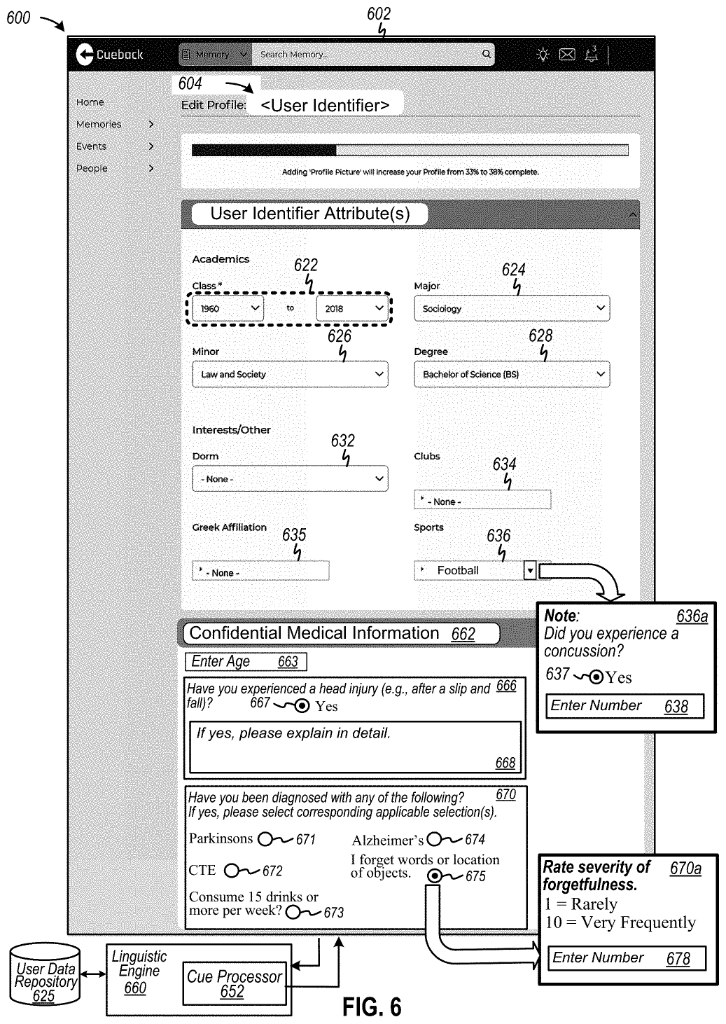

FIG. 6 is an example of a user interface to receive one or more prompts to identify one or more user attributes that may be relevant to determine a cognitive state, according to some examples. Diagram 600 depicts a user interface 602 configured to exchange data with a linguistic engine 660 to retrieve and analyze user-related attributes that may influence a manner in which a user speaks or writes from which linguistic samples may be extracted to perform cognitive analysis. User interface 602 includes of field 604 in which to accept data representing a user identifier, such as a name, user name, user account identifier, or the like. In some cases, linguistic engine 660 may analyze a name to determine whether a gender or sex-specific name may likely indicate whether a user is a male or female, a fact that may influence determinations of a cognitive state.

Further, user interface 602 includes user inputs, as prompts, to identify user-related attributes relating to, for example, a user's college experience. User input 622 may be configured to receive data selecting a date range during which the user attended a college. From this input, linguistic engine 660 may be configured to approximate an age based on years attending a 4-year college institution. Age-related information may be desired as it may influence a cognitive state of a user.

User interface 602 also includes a user input 624 configured to identify data representing a degree (e.g., a major or a specific area of specialized education), a user input 626 configured to identify data representing a degree (e.g., a minor or specific curriculum directed to a secondary or complementary area of education), and a user input 628 configured to receive data identifying a degree. From these inputs, linguistic engine 660 may be configured to approximate an education level of a user. Education level-related information may be desired as education may affect evaluation of a cognitive state.

Further, additional user attribute information may be identified, for example, via user input 632, which is configured to identify data representing a name of a dorm. Also, user input 634 may be configured to receive data representing one or more clubs, and user input 635 may be configured to receive data representing a Greek affiliation (e.g., a fraternity or sorority). In some cases, linguistic engine 660 may identify a dorm name and/or a Greek affiliation as either a fraternity or sorority to determine a gender or sex of a user because evaluations of a cognitive state based on linguistic samples may influenced by gender.

User input 636 may be configured to receive data representing one or more sporting activities a user participated in during college or in high school. Concussions and other head trauma may be more prevalent in full-contact sports, such as football and rugby, as well as soccer (e.g., due to head-butting a soccer ball). Thus, acquiring data confirming participation in such sports may be useful in determining a likelihood of cognitive deficiency. Responsive to selecting input 636, cue processor 652 may be configured to present a portion of a user interface 636a to provide user inputs 637 and 638 to ascertain whether a user has experienced one or more concussions, the effects of which may be discernible in evaluations of a user's spoken or written linguistic sample.

User interface 602 may also include additional user inputs directed to confidential medical information 662, which provides information that may be influential factors in evaluating cognitive states of a user and may provide a basis for modifying a weighting factor value, at least in some examples. User input 663 provides a field to explicitly receive data representing an age of user, and user inputs 667 and 668 of user interface portion 666 are configured to receive data to determine whether a user experienced a head injury. User interface portion 670 is configured to retrieve information relating to user diagnoses and other pertinent user conditions or attributes.

For example, user inputs 671, 672, and 674 requests whether a user has been diagnosed with Parkinson's disease, CTE, and Alzheimer's disease, respectively. Also, user input 673 is configured to confirm whether a user is a heavy drinker (e.g., an alcoholic), a history of which may correlate to cognitive declination. User input 675 provides for a self-assessment as to memory recall capabilities. If selected, cue processor 652 may be configured to present the user interface portion 670a to provide field 678, as a user input, in which a user may provide a self-assessed degree of “forgetfulness” or frequency of experiencing gaps in memory recall. In view of the foregoing, linguistic engine 660 may be configured to assess the above-described user attributes or conditions that may influence the formation of linguistic samples due to cognitive abilities. Such data may be stored in user data repository 625 for subsequent analysis.

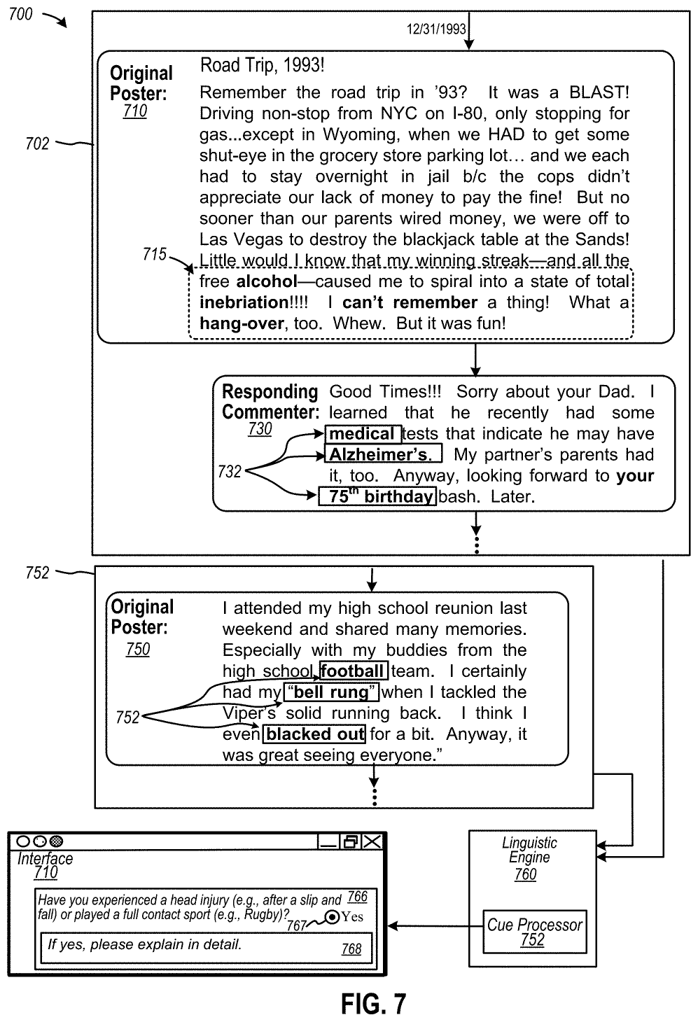

FIG. 7 is an example of a linguistic engine configured to analyze portions of the language to determine user attributes relevant to ascertain a cognitive state, according to some examples. Diagram 700 depicts user interface portions 702 and 752 each including one or more subsets of text in long form. In the example shown, data entry into interface portion 702 and 752 may be inputted into or transmitted to an on-line, cloud-based social archival platform configured to facilitate collaborative memory reinforcement, among other things.

Linguistic engine 760 may be configured to analyze each of text entries 710, 730, and 750 to determine whether any cognitive-related user attributes may be identified or otherwise predicted. For example, linguistic engine 760 may be configured to identify a portion of text 715 that includes terms “alcohol,” “inebriation,” “can't remember,” and “hang-over.” Based on these terms and the relative degree of relevancy to an activity of drinking, linguistic engine 760 may be configured to determine a likelihood that a user that posted text entry 710 may be inclined to consume excessive amounts of alcohol, at least during some points in time.

Also, linguistic engine 760 may be configured to identify portions of text 732 that includes terms “medical,” “Alzheimer's,” and “75th birthday.” Based on these terms, linguistic engine 760 may be configured to determine a likelihood that a user may be at risk of experiencing cognitive deficiencies due to age or disease (e.g., due to familial genetics).

Further, linguistic engine 760 may be configured to identify portions of text 732 that includes terms “football,” “bell rung,” and “blacked out.” Based on these terms, linguistic engine 760 may be configured to determine a likelihood that a user may be at risk of experiencing cognitive deficiencies due to past activities or head trauma.

In some examples, linguistic engine 760 may access data that may have yet to be entered into a user interface, such as interface 602 of FIG. 6, to establish certain user attributes or conditions. If a user has yet to access or input data into interface 602, then linguistic engine 760 of FIG. 7 may be configured to analyze text and identify risk factors that might influence cognitive integrity. As such, cue processor 752 may be configured to present an interface portion 766 in interface 710. Interface portion 766 includes user inputs 767 and 768 configured to receive data to validate predictions of certain user attributes based on text entries 710, 730, and 750. Note that the above-described examples are merely exemplary, and linguistic engine 760 may be configured to analyze any text portion to determine or predict any user attribute or condition for predicting a cognitive state.

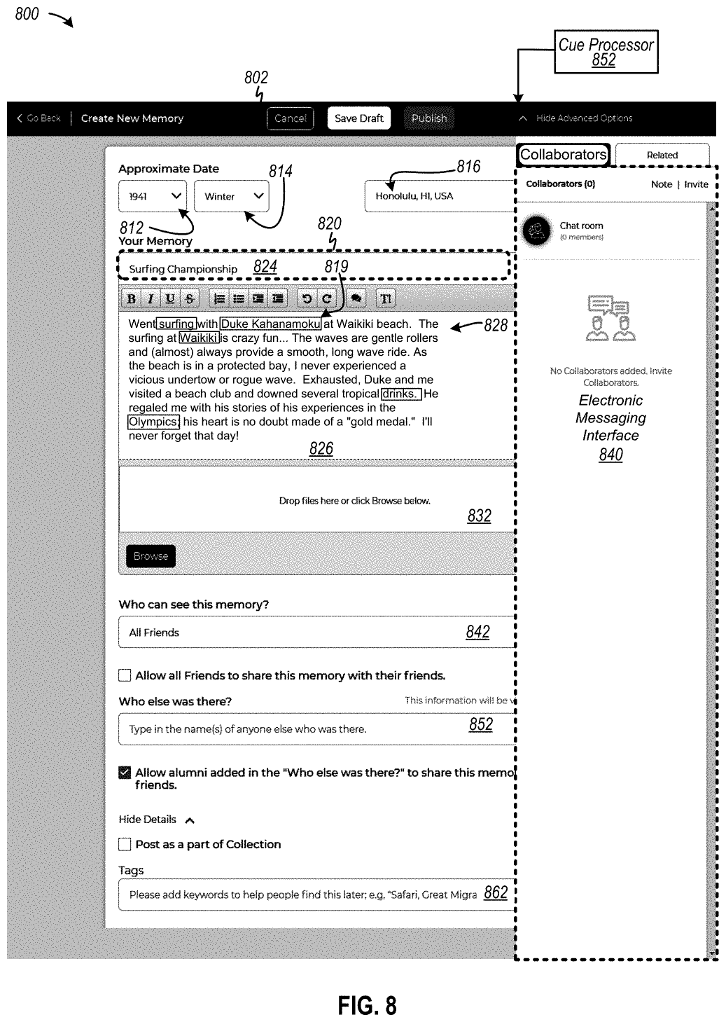

FIG. 8 is an example of a user interface to analyze data entry of a social media page to identify attributes to evaluate cognitive abilities of a user, according to some examples. Diagram 800 depicts a user interface 802 interface configured to exchange data with, for example, a cue processor 852, which may be configured to generate user inputs (as prompts) for user interface 802. User inputs 812 and 814 may be configured to receive data to identify an approximate time or duration of time of a memorable event. As shown, user input 812 may be configured to receive data representing a year (or any other date), and user input 814 may be configured to receive a “season” associated with a recollection should a user be uncertain of a particular month or day. User input 816 may be configured to receive data representing a geographic location at which a recollection occurred. Here, an activity “surfing championship” may be entered into field 824 of a entered text 820. User input 832 may be configured to receive content, such as images, audio files, or any other stimuli to provide context to evoke memories of, for example, any other use who may desire to collaborate on forming recollection collaboratively. Note that content, such as images and audio file may be tagged with metadata, such as names of people in a photo. Such information may be used to detect whether a user may correctly remember each name of a person tagged in a photo, thereby facilitating a means with which to evaluate a user's memory recall capabilities.

A description 828 of an event may be entered into editor portion 826 of interface 802. Note, too, that cue processor 852 may be configured to capture text in description 828 as terms that may be used to link with other portions of text to, for example, perform memory recall analysis (e.g., by checking consistency in use of terms). Thus, “surfing,” “Duke Kahanamoku,” and “Waikiki” may be stored in a data model repository for predicting subsequent cues for presentation as well as testing memory recall or other implanting any other algorithm to compute a linguistic measure value. The term “drinks” may be identified as possible evidence of an inclination to drink alcohol with frequency, which may affect cognitive abilities. Further, text in description 828 may be considered as a “linguistic sample” of long form text that may be analyzed to determine various linguistic measures, such as P-density values, D-Level values, and the like.

User input 842 may be configured to restrict access, modification, or propagation of recollection 820 based on, for example, a type of collaborative user for which permission is granted. Here, all “friends” are granted access. User input 852 may be configured to receive data representing specific users that may be associated with recollection 820 and who may have knowledge to “challenge” inconsistent usage of terms (i.e., due to lapses in memory). User input 862 may be configured to receive data representing user-provided tags or metadata, which may be used for forming cues or prompts. Additionally, interface 802 may include an electronic messaging interface 840 accessible to request information from other collaborative users in-situ (i.e., an electronic message communication channel may be established during presentation or contemporaneous with presentation of user interface 802, which may be configured to form a recollection). In particular, a user forming a recollection titled “surfing championship” in field 824 may wish to contact some other user or person in real-time (or nearly real-time) to receive immediate feedback or clarification based on another's experience or memory recall capabilities. In response, other collaborative users may provide supplemental information via interface 840, which, in turn, may be added to supplement the recollection.

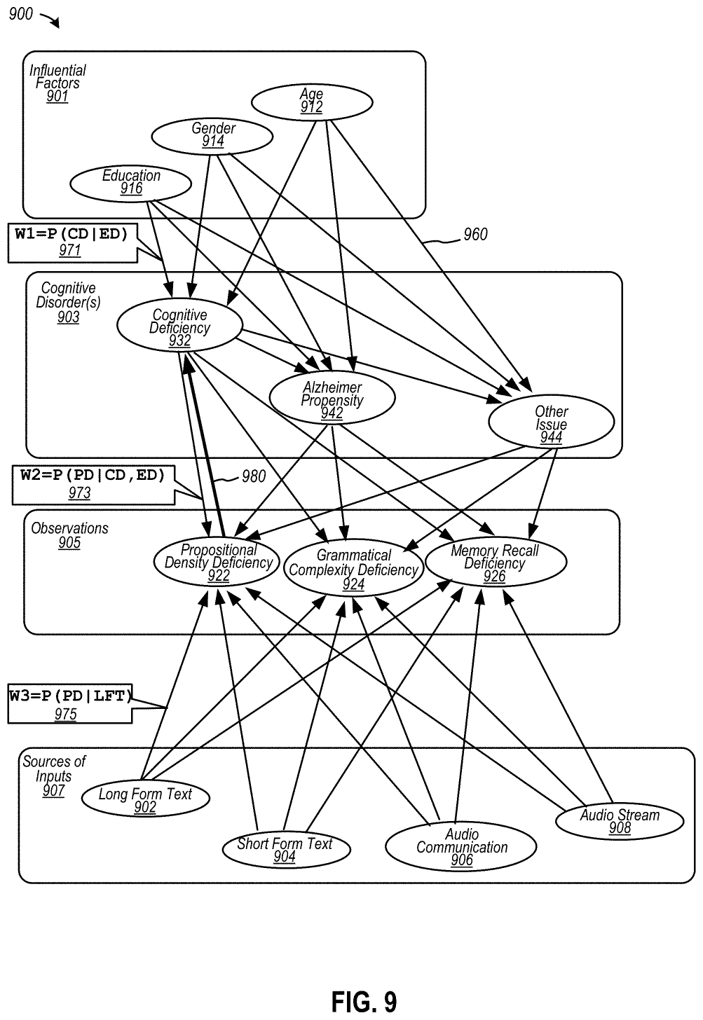

FIG. 9 is a diagram depicting an example of a probabilistic prediction model to infer a cognitive state as a function of characterizing linguistic samples, according to some examples. Diagram 900 depicts a probabilistic network graph configured to model causation of events as a function of probabilities and conditional dependency. In some examples, a model of diagram 900 may be implemented as a Bayesian network model. As shown, an example of a model includes a first subset of influential factors 901 as variables that may influence language production as a function of age 921, gender 914 (or sex), and education level 916. A second subset of variables, which may be considered hidden, are cognitive disorders 903. This subset of variables include a general cognitive state in which a cognitive deficiency 932 may exist. Another variable is an event that a user may be afflicted with Alzheimer's disease 942 (e.g., may have early stages of Alzheimer's disease or a propensity to subsequently be diagnosed as such). A variable identified as “other issue(s)” 944 may include events or occurrences associated with the presence of one or more other cognitive disorders, such as CTE, Parkinson's disease, etc., or other causes of cognitive degeneration, such as alcoholism, head trauma, etc.

Observations 905 include variables that may be observed (or measured) as evidence of a cognitive disorder. For example, a propositional density deficiency value 922, a grammatical complexity deficiency value 924, a memory recall deficiency value 926, and any other linguistic measure value may be computed to generate data representative to characterize language production. Various characterizations or attributes of language productions may be determined by computing any number of linguistic measure values that are likely caused by a specific cognitive state (e.g., a cognitive deficiency or impairment).

Sources of inputs 907 represent variables that may relate to sources of linguistic samples from which to generate linguistic measure values in observations 905. As shown, sources of inputs 907 may provide linguistic samples extracted from one or more of long form text entry data 902, short form text entry data 904 (e.g., SMS text messages), audio communication data 906 (e.g., oral speech captured during cellular phone calls), and audio streaming data 908 (from which a linguistic sample may be extracted from, for example, audio generated by a voice-activated microphone on a mobile phone).

According to some examples, each source of input 907 may influence formation of language production differently. Thus, linguistic samples may be formed to generate different linguistic measure values. For instance, consider that long form text entries 902 may be more reliable or may produce more accurate linguistic samples, which, in turn, may provide more precise linguistic measure values. Also, long form text-based linguistic samples may be generated at a rate at which a user, especially an elderly user, is personally comfortable. By contrast, audio-based linguistic samples 906 derived from a telephone conversation with another person may produce anxiety and cause formation of less complex sentences, for example. Thus, a weighting factor value for long term text data usage may be relatively higher than a weighting factor value associated with linguistic samples developed from a telephone conversation. In some examples, such weighting factor values are a function of, or correlatable to, probabilities.

Edges 906 may represent probabilities, such as conditional probabilities, that may provide a “weighting factor value” or at least a basis from which to determine a weighting factor value described in, for example, FIG. 4. To illustrate, consider the following example. As shown, an event of having a cognitive deficiency (“CD”) 932 may be conditional on a probability 971 of having a specific education level (“ED”). Having a cognitive deficiency (“CD”) 932 may be due to calculating a specific value of a linguistic measure (e.g., specific value of P-density 922 (“PD”)), which may be observed. Thus, a probability 973 of detecting a specific value of P-density may be conditioned on having a cognitive deficiency (“CD”) 932, which, in turn, may be conditioned on having a specific education level (“ED”). Also, in some cases, a specific value of P-density 922 (“PD”) may be conditioned on a probability 975 responsive to using a linguistic sample originating from a source of long form text (“LFT”) 902. Further, “retrospective propagation” may be used to determine an inverse of predictive propagation to determine probability 980. That is, a probability of having a level of cognitive deficiency 932 may be based, at least in part, on an event of demonstrating a certain linguistic measure value, such as P-density 922. The above description of probabilities omits discussion of other conditional probabilities so as not to obscure a description of the model.

Note that a model shown in diagram 900 is but one example, and is not intended to be limiting. Any other model may be used, such as a model based on support vector machines (“SVMs”), various types of neural networks (e.g., convolutional neural networks (“CNN”), recurrent neural networks (“RNN”), artificial neural networks (“ANN”), and the like), various regression techniques, various k-means computations, or any other like algorithms. Further, a self-learning Bayesian network model of diagram 900 may be replaced or supplemented by any machine learning and/or deep learning algorithms, any of which may be probabilistic or otherwise, according to some examples. Additionally, the model shown in FIG. 900 may be varied to produce any other configurations of Bayesian networks and variants thereof.

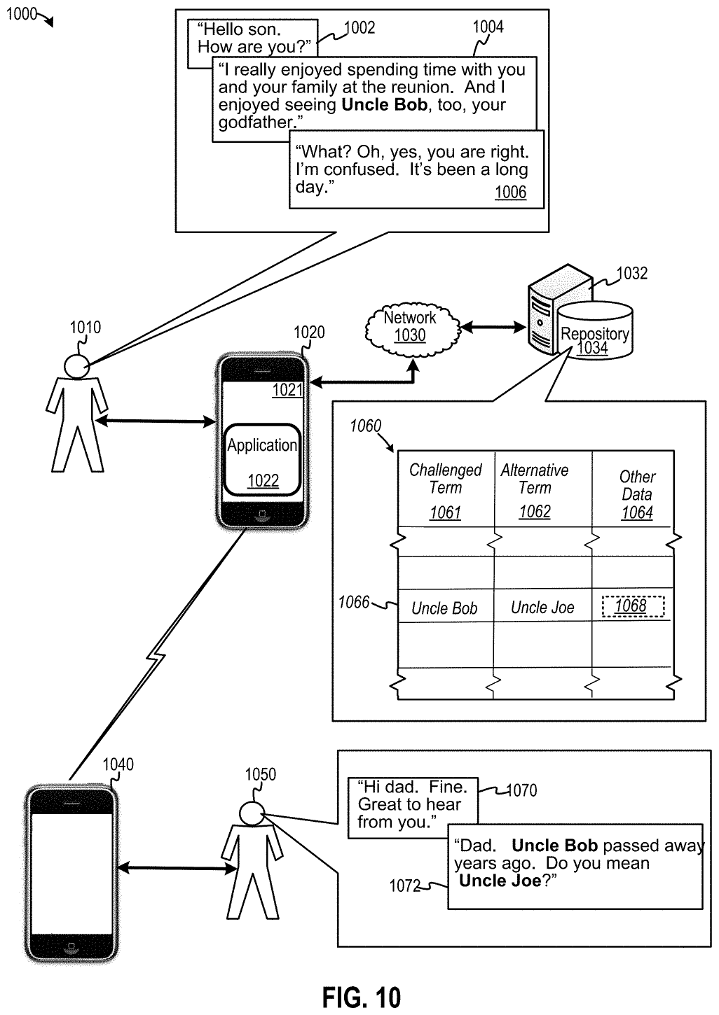

FIG. 10 is a diagram depicting an example of a computing platform configured to implement a linguistic engine to detect and manage instances of a challenged term, according to some examples. A challenged term may refer to a word, fact, concept, or idea that a user may express to, for example, another person, who, in turn, may challenge that word, fact, concept, or idea. Lapses in recalling memory may increase with age as well as increasingly detectable symptoms of a cognitive deficiency, such as Alzheimer's disease. So, at least in some cases, increases in detected challenged terms may be indicative of cognitive impairment.

In the example shown, a linguistic engine may be implemented using a processor in a computing device 1032, which is coupled to a repository 1034. Repository 1034 may include a data arrangement 1060 that stores data representing challenged terms 1061, alternative terms 1062, and other data 1064, if applicable. Consider user 1010 may be engaged in a telephonic conversation with user 1050 via mobile computing device 1040. Mobile device 1020 associated with user 1010 may include an application 1022 that may be configured to passively transmit at least audio from user 1010 (received from a phone's microphone) via a network 1030 to computing device 1032.

Further to the example shown, a conversation between user 1010 and user 1050 may be as follows. “Hello son. How are you?,” which may be captured as a linguistic sample 1002. User 1050 responds, “Hi dad. Fine. Great to hear from you.” (Linguistic sample 1070). User 1010 continues, “I really enjoyed spending time with you and your family at the reunion. And I enjoyed seeing Uncle Bob, too, your godfather.” (Linguistic sample 1004). User 1050 again responds, “Dad. Uncle Bob passed away years ago. Do you mean uncle Joe?” (Linguistic sample 1072). User 1010 replies, “What? Oh, yes, you are right I'm confused. It's been a long day.” (Linguistic sample 1006).

A linguistic engine may convert the conversation from speech into text and perform any number of computer-based language processing techniques to analyze the linguistic samples. The linguistic engine can identify from the conversation that the term “Uncle Bob” was challenged, to which an alternative term “Uncle Joe” was proffered. This conflict is stored as data record 1066. The linguistic engine subsequently may track occurrences of challenged terms, and a value representing a memory recall deficiency may be generated (e.g., as a function of the frequency of challenged terms, as well as other factors (such as the type of word or fact that may be identified and stored as data 1068)). The above is but one example of measuring memory recall deficiencies, and any other process or computer algorithm may be implemented. For example, a linguistic engine may be configured to automatically detect a challenged term and compute an alternative term for presentation to user 1010 for clarification, such as in prompt 162 of FIG. 1A.

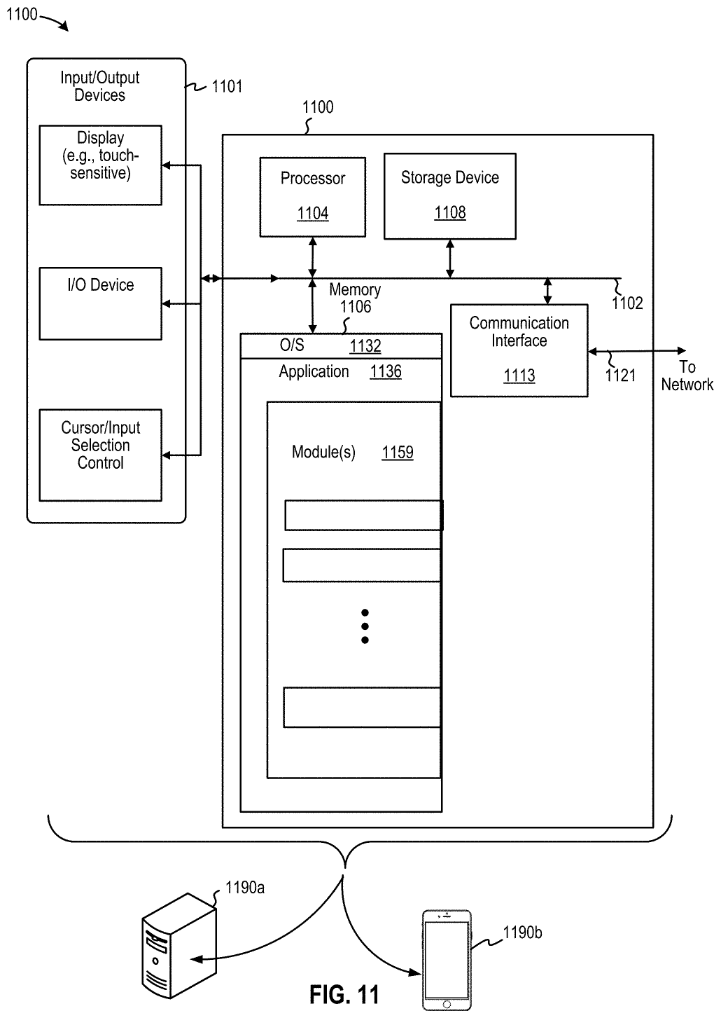

FIG. 11 illustrates examples of various computing platforms configured to provide various functionalities to components of a collaborative recollection engine and/or a linguistic engine, according to various embodiments. In some examples, computing platform 1100 may be used to implement computer programs, applications, methods, processes, algorithms, or other software, as well as any hardware implementation thereof, to perform the above-described techniques.

In some cases, computing platform 1100 or any portion (e.g., any structural or functional portion) can be disposed in any device, such as a computing device 1190a, mobile computing device 1190b, and/or a processing circuit in forming structures and/or functions of an above-described apparatus, system, platform or device, according to various examples described herein.

Computing platform 1100 includes a bus 1102 or other communication mechanism for communicating information, which interconnects subsystems and devices, such as processor 1104, system memory 1106 (e.g., RAM, etc.), storage device 1108 (e.g., ROM, etc.), an in-memory cache (which may be implemented in RAM 1106 or other portions of computing platform 1100), a communication interface 1113 (e.g., an Ethernet or wireless controller, a Bluetooth controller, NFC logic, etc.) to facilitate communications via a port on communication link 1121 to communicate, for example, with a computing device, including mobile computing and/or communication devices with processors, including database devices (e.g., storage devices configured to store atomized datasets, including, but not limited to triplestores, etc.). Processor 1104 can be implemented as one or more graphics processing units (“GPUs”), as one or more central processing units (“CPUs”), such as those manufactured by Intel® Corporation, or as one or more virtual processors, as well as any combination of CPUs and virtual processors. Computing platform 1100 exchanges data representing inputs and outputs via input-and-output devices 1101, including, but not limited to, keyboards, mice, audio inputs (e.g., speech-to-text driven devices), user interfaces, displays, monitors, cursors, touch-sensitive displays, LCD or LED displays, and other I/O-related devices.

Note that in some examples, input-and-output devices 1101 may be implemented as, or otherwise substituted with, a user interface in a computing device associated with a user account identifier in accordance with the various examples described herein.

According to some examples, computing platform 1100 performs specific operations by processor 1104 executing one or more sequences of one or more instructions stored in system memory 1106, and computing platform 1100 can be implemented in a client-server arrangement, peer-to-peer arrangement, or as any mobile computing device, including smart phones and the like. Such instructions or data may be read into system memory 1106 from another computer readable medium, such as storage device 1108. In some examples, hard-wired circuitry may be used in place of or in combination with software instructions for implementation. Instructions may be embedded in software or firmware. The term “computer readable medium” refers to any tangible medium that participates in providing instructions to processor 1104 for execution. Such a medium may take many forms, including but not limited to, non-volatile media and volatile media. Non-volatile media includes, for example, optical or magnetic disks and the like. Volatile media includes dynamic memory, such as system memory 1106.

Known forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can access data. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1102 for transmitting a computer data signal.

In some examples, execution of the sequences of instructions may be performed by computing platform 1100. According to some examples, computing platform 1100 can be coupled by communication link 1121 (e.g., a wired network, such as LAN, PSTN, or any wireless network, including WiFi of various standards and protocols, Bluetooth®, NFC, Zig-Bee, etc.) to any other processor to perform the sequence of instructions in coordination with (or asynchronous to) one another. Computing platform 1100 may transmit and receive messages, data, and instructions, including program code (e.g., application code) through communication link 1121 and communication interface 1113. Received program code may be executed by processor 1104 as it is received, and/or stored in memory 1106 or other non-volatile storage for later execution.

In the example shown, system memory 1106 can include various modules that include executable instructions to implement functionalities described herein. System memory 1106 may include an operating system (“O/S”) 1132, as well as an application 1136 and/or logic module(s) 1159. In the example shown in FIG. 11, system memory 1106 may include any number of modules 1159, any of which, or one or more portions of which, can be configured to facilitate any one or more components of a computing system (e.g., a client computing system, a server computing system, etc.) by implementing one or more functions described herein.

The structures and/or functions of any of the above-described features can be implemented in software, hardware, firmware, circuitry, or a combination thereof.

Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit.

According to some embodiments, the term “module” can refer, for example, to an algorithm or a portion thereof, and/or logic implemented in either hardware circuitry or software, or a combination thereof. These can be varied and are not limited to the examples or descriptions provided.

In some embodiments, modules 1159 of FIG. 20, or one or more of their components, or any process or device described herein, can be in communication (e.g., wired or wirelessly) with a mobile device, such as a mobile phone or computing device, or can be disposed therein.

In some cases, a mobile device, or any networked computing device (not shown) in communication with one or more modules 1159 or one or more of its/their components (or any process or device described herein), can provide at least some of the structures and/or functions of any of the features described herein. As depicted in the above-described figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in any of the figures can represent one or more algorithms. Or, at least one of the elements can represent a portion of logic including a portion of hardware configured to provide constituent structures and/or functionalities.

For example, modules 1159 or one or more of its/their components, or any process or device described herein, can be implemented in one or more computing devices (i.e., any mobile computing device, such as a wearable device, such as a hat or headband, or mobile phone, whether worn or carried) that include one or more processors configured to execute one or more algorithms in memory. Thus, at least some of the elements in the above-described figures can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit.

For example, modules 1159 or one or more of its/their components, or any process or device described herein, can be implemented in one or more computing devices that include one or more circuits. Thus, at least one of the elements in the above-described figures can represent one or more components of hardware. Or, at least one of the elements can represent a portion of logic including a portion of a circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

As used herein, “system” may refer to or include the description of a computer, network, or distributed computing system, topology, or architecture using various computing resources that are configured to provide computing features, functions, processes, elements, components, or parts, without any particular limitation as to the type, make, manufacturer, developer, provider, configuration, programming or formatting language, service, class, resource, specification, protocol, or other computing or network attributes. As used herein, “software” or “application” may also be used interchangeably or synonymously with, or refer to a computer program, software, program, firmware, or any other term (e.g., engine) that may be used to describe, reference, or refer to a logical set of instructions that, when executed, performs a function or set of functions within a computing system or machine, regardless of whether physical, logical, or virtual and without restriction or limitation to any particular implementation, design, configuration, instance, or state. Further, “platform” may refer to any type of computer hardware (hereafter “hardware”) or software, or any combination thereof, that may use one or more local, remote, distributed, networked, or computing cloud (hereafter “cloud”)-based computing resources (e.g., computers, clients, servers, tablets, notebooks, smart phones, cell phones, mobile computing platforms or tablets, and the like) to provide an application, operating system, or other computing environment, such as those described herein, without restriction or limitation to any particular implementation, design, configuration, instance, or state. Distributed resources such as cloud computing networks (also referred to interchangeably as “computing clouds,” “storage clouds,” “cloud networks,” or, simply, “clouds,” without restriction or limitation to any particular implementation, design, configuration, instance, or state) may be used for processing and/or storage of varying quantities, types, structures, and formats of data, without restriction or limitation to any particular implementation, design, or configuration.

As used herein, data may be stored in various types of data structures including, but not limited to databases, data repositories, data warehouses, data stores, or other data structures configured to store data in various computer programming languages and formats in accordance with various types of structured and unstructured database schemas such as SQL, MySQL, NoSQL, DynamoDB™, etc. Also applicable are computer programming languages and formats similar or equivalent to those developed by data facility and computing providers such as Amazon® Web Services, Inc. of Seattle, Wash., FMP, Oracle®, Salesforce.com, Inc., or others, without limitation or restriction to any particular instance or implementation. DynamoDB™, Amazon Elasticsearch Service, Amazon Kinesis Data Streams (“KDS”)™, Amazon Kinesis Data Analytics, and the like, are examples of suitable technologies provide by Amazon Web Services (“AWS”).

Further, references to databases, data structures, or any type of data storage facility may include any embodiment as a local, remote, distributed, networked, cloud-based, or combined implementation thereof. For example, social networks and social media (hereafter “social media”) using different types of devices may generate (i.e., in the form of posts (which is to be distinguished from a POST request or call over HTTP) on social networks and social media) data in different forms, formats, layouts, data transfer protocols, and data storage schema for presentation on different types of devices that use, modify, or store data for purposes such as electronic messaging, audio or video rendering, content sharing, or like purposes. Data may be generated in various formats such as text, audio, video (including three dimensional, augmented reality (“AR”), and virtual reality (“VR”), or others, without limitation, for use on social networks, social media, and social applications (hereafter “social media”) such as Twitter® of San Francisco, Calif., Snapchat® as developed by Snap® of Venice, Calif., Messenger as developed by Facebook®, WhatsApp®, or Instagram® of Menlo Park, Calif., Pinterest® of San Francisco, Calif., LinkedIn® of Mountain View, Calif., and others, without limitation or restriction.

In some examples, data may be formatted and transmitted (i.e., transferred over one or more data communication protocols) between computing resources using various types of data communication and transfer protocols such as Hypertext Transfer Protocol (“HTTP”), Transmission Control Protocol (“TCP”)/Internet Protocol (“IP”), Internet Relay Chat (“IRC”), SMS, text messaging, instant messaging (“IM”), File Transfer Protocol (“FTP”), or others, without limitation. As described herein, disclosed processes implemented as software may be programmed using Java®, JavaScript®, Scala, Python™, XML, HTML, and other data formats and programs, without limitation. Disclosed processes herein may also implement software such as Streaming SQL applications, browser applications (e.g., Firefox™) and/or web applications, among others. In some example, a browser application may implement a

JavaScript framework, such as Ember.js, Meteor.js, ExtJS, AngularJS, and the like. References to various layers of an application architecture (e.g., application layer or data layer) may refer to a stacked layer application architecture such as the Open Systems Interconnect (“OSI”) model or others.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

1. A method comprising:
receiving data representing alpha-numeric text forming words constituting one or more sentences;
classifying one or more words based on a syntactic function to form classified words at a processor configured to analyze the data representing the alpha-numeric text;
identifying subsets of words constituting portions of sentences to form identified portions of sentences;
identifying a source of the alpha-numeric text;
executing instructions at the processor to characterize one or more subsets of the alpha-numeric text and the identified portions of sentences to form characterized data associated with data representing a value indicative of a cognitive state, the characterized data being a function of the source of the alpha-numeric text; and
generating an electronic message as a notification describing one of a subset of cognitive states.

2. The method of claim 1 wherein identifying the source of the alpha-numeric text further comprises:
identifying multiple sources of the alpha-numeric text.

3. The method of claim 2 wherein executing instructions at the processor to form the characterized data as a function of the source comprises:
implementing a weighting factor associated corresponding to each of the multiple sources of the alpha-numeric text.

4. The method of claim 1 wherein the value indicative of the cognitive state includes one or more cognitive states including Alzheimer's disease, chronic traumatic encephalopathy (“CTE”), and age-related cognitive decline.

5. The method of claim 1 wherein executing the instructions at the processor to form characterized data comprises:
computing a linguistic measure to form the value indicative of the cognitive state.

6. The method of claim 5 wherein computing a linguistic measure comprises:
calculating data representing a proposition density value per unit of text, the proposition density value being indicative of a rate of ideas expressed per the unit of text.

7. The method of claim 6 further comprising:
correlating the proposition density value as the value indicative of the cognitive state to a subset of proposition density values to determine the cognitive state; and
identifying a cognitive disorder based the cognitive state.

8. The method of claim 5 wherein computing a linguistic measure comprises:
calculating data representing a developmental level value per unit of text, the developmental level value being indicative of grammatical complexity per the unit of text.

9. The method of claim 8 further comprising:
correlating the developmental level value as the value indicative of the cognitive state to a subset of developmental level values to determine the cognitive state; and
identifying a cognitive disorder based the cognitive state.

10. The method of claim 1 further comprising:
receiving data representing a subset of alpha-numeric text as a challenge response including one or more words at conflict, the challenge response indicative of an issue of memory recall.

11. The method of claim 10 further comprising:
identifying a challenge term and an alternative term at conflict; and
modifying a value associated corresponding by an amount indicative of the issue of memory recall.

12. The method of claim 10 further comprises:
causing presentation of a portion of a user interface to include a user input to confirm whether a challenge term ought to be replaced by an alternative term.

13. An apparatus comprising:
a memory including executable instructions; and
a processor, responsive to executing the instructions, is configured to:
receive data representing alpha-numeric text forming words constituting one or more sentences;
classify one or more words based on a syntactic function to form classified words at a processor configured to analyze the data representing the alpha-numeric text;
identify subsets of words constituting portions of sentences to form identified portions of sentences;
identify a source of the alpha-numeric text;
execute instructions at the processor to characterize one or more subsets of the alpha-numeric text and the identified portions of sentences to form characterized data associated with data representing a value indicative of a cognitive state, the characterized data being a function of the source of the alpha-numeric text; and
generate an electronic message as a notification describing one of a subset of cognitive states.

14. The apparatus of claim 13 wherein a subset of the instructions further causes the processor to:
identify the source of the alpha-numeric text as multiple sources of the alpha-numeric text; and
implement a weighting factor associated corresponding to each of the multiple sources of the alpha-numeric text.

15. The apparatus of claim 13 wherein the value indicative of the cognitive state includes one or more cognitive states including Alzheimer's disease, chronic traumatic encephalopathy (“CTE”), and age-related cognitive decline.

16. The apparatus of claim 13 wherein a subset of the instructions further causes the processor to:
compute a linguistic measure to form the value indicative of the cognitive state.

17. The apparatus of claim 16 wherein the subset of the instructions that causes the processor to compute the linguistic measure comprises another subset of the instructions configured to:
calculate data representing a proposition density value per unit of text, the proposition density value being indicative of a rate of ideas expressed per the unit of text;
correlate the proposition density value as the value indicative of the cognitive state to a subset of proposition density values to determine the cognitive state; and
identify a cognitive disorder based the cognitive state.

18. The apparatus of claim 16 wherein the subset of the instructions that causes the processor to compute the linguistic measure comprises another subset of the instructions configured to:
calculate data representing a developmental level value per unit of text, the developmental level value being indicative of grammatical complexity per the unit of text;
correlate the developmental level value as the value indicative of the cognitive state to a subset of developmental level value to determine the cognitive state; and
identify a cognitive disorder based the cognitive state.

19. The apparatus of claim 13 wherein a subset of the instructions further causes the processor to:
receive data representing a subset of alpha-numeric text as a challenge response including one or more words at conflict, the challenge response indicative of an issue of memory recall.

20. The apparatus of claim 19 further comprising another subset of instructions configured to cause the processor to:
identify a challenge term and an alternative term at conflict; and
modify a value associated corresponding by an amount indicative of the issue of memory recall.
